(19) United States
(12) Patent Application Publication
(10) Pub. No.: US 2010/0292266 A1
(43) Pub. Date: Nov. 18, 2010

(54) Oxazolyl Piperidine Modulators of Fatty Acid Amide Hydrolase

(76) Inventors: Richard Apodaca, San Diego, CA (US); James Guy Breitenbucher, Escondido, CA (US); Alison L. Chambers, San Diego, CA (US); Mark Seierstad, Escondido, CA (US); Wei Xiao, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/227,756

(22) PCT Filed: May 25, 2007
(86) PCT No.: PCT/US2007/012631

§ 371 Date: Nov. 25, 2008

Related U.S. Application Data
(60) Provisional application No. 60/808,723, filed on May 26, 2006.

(51) Int. Cl. A61K 31/454; C07D 413/06; C07D 413/14; C07D 471/04; A61K 31/4545; C07D 491/056; A61K 31/4709; A61P 29/00; A61P 25/00; A61P 35/00; A61P 1/08; A61P 1/04

(52) U.S. Cl. 514/291; 546/206; 514/319; 546/209; 514/326; 546/197; 514/321; 546/201; 514/323; 546/122; 514/300; 546/199; 514/322; 546/193; 514/318; 546/90; 546/165; 514/314

(57) ABSTRACT

Certain oxazolyl piperidine compounds are described, which are useful as FAAH inhibitors. Such compounds may be used in pharmaceutical compositions and methods for the treatment of disease states, disorders, and conditions mediated by fatty acid amide hydrolase (FAAH) activity. Thus, the compounds may be administered to treat, e.g., anxiety, pain, inflammation, sleep disorders, eating disorders, or movement disorders (such as multiple sclerosis).

### CROSS-REFERENCE TO RELATED APPLICATION

This application claims priority to U.S. Provisional Application No. 60/808,723, filed May 26, 2006.

### FIELD OF THE INVENTION

The present invention relates to certain oxazolyl piperidine compounds, pharmaceutical compositions containing them, and methods of using them for the treatment of disease states, disorders, and conditions mediated by fatty acid amide hydrolase (FAAH) activity.

### BACKGROUND OF THE INVENTION

Medicinal benefits have been attributed to the cannabis plant for centuries. The primary bioactive constituent of cannabis is Δ⁹-tetrahydro-cannabinol (THC). The discovery of THC eventually led to the identification of two endogenous cannabinoid receptors responsible for its pharmacological actions, namely CB₁ and CB₂ (Goya, Exp. Opin. Ther. Patents 2000, 10, 1529). These discoveries not only established the site of action of THC, but also inspired inquiries into the endogenous agonists of these receptors, or “endocannabinoids”. The first endocannabinoid identified was the fatty acid amide anandamide (AEA). AEA itself elicits many of the pharmacological effects of exogenous cannabinoids (Piomelli, Nat. Rev. Neurosci. 2003, 4(11), 873).

The catabolism of AEA is primarily attributable to the integral membrane bound protein fatty acid amide hydrolase (FAAH), which hydrolyzes AEA to arachidonic acid. FAAH was characterized in 1996 by Cravatt and co-workers (Cravatt, Nature 1996, 384, 83). It was subsequently determined that FAAH is additionally responsible for the catabolism of a large number of important lipid signaling fatty acid amides including: another major endocannabinoid, 2-arachidonoylglycerol (2-AG) (Science 1992, 258, 1946-1949); the sleep-inducing substance, oleamide (OEA) (Science 1995, 268, 1506); the appetite-suppressing agent, N-oleoylethanolamine (Rodriguez de Fonesca, Nature 2001, 414, 209); and the anti-inflammatory agent, palmitoylethanolamide (PEA) (Lambert, Curr. Med. Chem. 2002, 9(6), 663).

Small-molecule inhibitors of FAAH should elevate the concentrations of these endogenous signaling lipids and thereby produce their associated beneficial pharmacological effects. There have been some reports of the effects of various FAAH inhibitors in pre-clinical models.

In particular, two carbamate-based inhibitors of FAAH were reported to have analgesic properties in animal models. In rats, BMS-1 (see WO 02/087569), which has the structure shown below, was reported to have an analgesic effect in the Chung spinal nerve ligation model of neuropathic pain, and the Hargraves test of acute thermal nociception. URB-597 was reported to have efficacy in the zero plus maze model of anxiety in rats, as well as analgesic efficacy in the rat hot plate and formalin tests (Kathuria, Nat. Med. 2003, 9(1), 76). The sulfonyifluoride AM374 was also shown to significantly reduce spasticity in chronic relapsing experimental autoimmune encephalomyelitis (CREAE) mice, an animal model of multiple sclerosis (Baker, FASEB J. 2001, 15(2), 300).

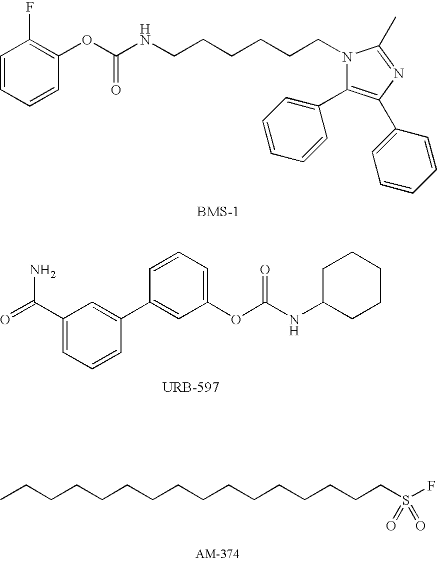

In addition, the oxazolopyridine ketone OL-135 is reported to be a potent inhibitor of FAAH, and has been reported to have analgesic activity in both the hot plate and tail emersion tests of thermal nociception in rats (WO 04/033652).

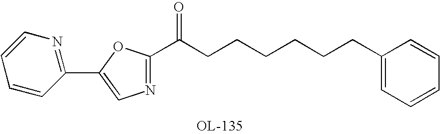

Results of research on the effects of certain exogenous cannabinoids has elucidated that a FAAH inhibitor may be useful for treating various conditions, diseases, disorders, or symptoms. These include pain, nausea/emesis, anorexia, spasticity, movement disorders, epilepsy and glaucoma. To date, approved therapeutic uses for cannabinoids include the relief of chemotherapy-induced nausea and emesis among patients with cancer and appetite enhancement in patients with HIV/AIDs who experience anorexia as a result of wasting syndrome. Two products are commercially available in some countries for these indications, namely, dronabinol (Marinol®) and nabilone.

Apart from the approved indications, a therapeutic field that has received much attention for cannabinoid use is analgesia, i.e., the treatment of pain. Five small randomized controlled trials showed that THC is superior to placebo, producing dose-related analgesia (Robson, Br. J. Psychiatry 2001, 178, 107-115). Atlantic Pharmaceuticals is reported to be developing a synthetic cannabinoid, CT-3, a 1,1-dimethyl heptyl derivative of the carboxylic metabolite of tetrahydrocannabinol, as an orally active analgesic and anti-inflammatory agent. A pilot phase II trial in chronic neuropathic pain with CT-3 was reported to have been initiated in Germany in May 2002.

A number of individuals with multiple sclerosis have claimed a benefit from cannabis for both disease-related pain and spasticity, with support from small controlled trials (Svendsen, Br. Med. J. 2004, 329, 253). Likewise, various victims of spinal cord injuries, such as paraplegia, have reported that their painful spasms are alleviated after smoking marijuana. A report showing that cannabinoids appear to control spasticity and tremor in the CREAE model of multiple sclerosis demonstrated that these effects are mediated by CB₁ and CB₂ receptors (Baker, Nature 2000, 404, 84-87). Phase 3 clinical trials have been undertaken in multiple sclerosis and spinal cord injury patients with a narrow ratio mixture of tetrahydrocannabinol/cannabidiol (THC/CBD).

Reports of small-scale controlled trials have been conducted to investigate other potential commercial uses of cannabinoids have been made: Trials in volunteers have been reported to have confirmed that oral, injected and smoked cannabinoids produced dose-related reductions in intraocular pressure (IOP) and therefore may relieve glaucoma symptoms. Ophthalmologists have prescribed cannabis for patients with glaucoma in whom other drugs have failed to adequately control intraocular pressure (Robson, 2001).

Inhibition of FAAH using a small-molecule inhibitor may be advantageous compared to treatment with a direct-acting CB₁ agonist. Administration of exogenous CB₁ agonists may produce a range of responses, including reduced nociception, catalepsy, hypothermia, and increased feeding behavior. These four in particular are termed the “cannabinoid tetrad.” Experiments with FAAH −/− mice show reduced responses in tests of nociception, but did not show catalepsy, hypothermia, or increased feeding behavior (Cravatt, Proc. Natl. Acad. Scl. USA 2001, 98(16), 9371). Fasting caused levels of AEA to increase in rat limbic forebrain, but not in other brain areas, providing evidence that stimulation of AEA biosynthesis may be anatomically regionalized to targeted CNS pathways (Kirkham, Br. J. Pharmacol. 2002, 136, 550). The finding that AEA increases are localized within the brain, rather than systemic, suggests that FAAH inhibition with a small molecule could enhance the actions of AEA and other fatty acid amides in tissue regions where synthesis and release of these signaling molecules is occurring in a given pathophysiological condition (Piomelli, 2003).

In addition to the effects of a FAAH inhibitor on AEA and other endocannabinoids; inhibitors of FAAH's catabolism of other lipid mediators may be used in treating other therapeutic indications. For example, PEA has demonstrated biological effects in animal models of inflammation (Holt, et al. Br. J. Pharmacol.2005, 146, 467-476), immunosuppression, analgesia, and neuroprotection (Ueda, J. Biol. Chem. 2001, 276(38), 35552). Oleamide, another substrate of FAAH, induces sleep (Boger, Proc. Natl. Acad. Sci. USA 2000, 97(10), 5044; Mendelson, Neuropsychopharmacology 2001, 25, S36). Inhibition of FAAH has also been implicated in cognition (Vervel, et al. J. Pharmacot Exp. Ther. 2006, 317(1), 251-257) and depression (Gobbi, et al. Proc. Natl. Acad. Sci. USA 2005, 102(51), 18620-18625).

Thus, there is evidence that small-molecule FAAH inhibitors may be useful in treating pain of various etiologies, anxiety, multiple sclerosis and other movement disorders, nausea/emesis, eating disorders, epilepsy, glaucoma, inflammation, immunosuppression, neuroprotection, depression, cognition enhancement, and sleep disorders, and potentially with fewer side effects than treatment with an exogenous cannabinoid.

Various small-molecule FAAH modulators have been reported; e.g., in WO 04/033652, U.S. Pat. No. 6,462,054, U.S. Pat. No. 6,096,784, WO 99/26584, WO 97/49667, WO 96/09817, U.S. patent application Ser. No. 11/321,710 (Dec. 29, 2005), and U.S. patent application No. 11/251,317 (Oct. 14, 2005). Certain FAAH modulators are also described in U.S. Provisional Appl. No. 60/696,166, filed Jun. 30, 2005, and U.S. Provisional Appl. No. 60/738,248, filed Nov. 18, 2005. However, there remains a desire for potent FAAH modulators with suitable pharmaceutical properties.

### SUMMARY OF THE INVENTION

Certain oxazolyl piperidine derivatives have now been found to have FAAH-modulating activity. Thus, the invention is directed to the general and preferred embodiments defined, respectively, by the independent and dependent claims appended hereto, which are incorporated by reference herein.

In one general aspect the invention features a chemical entity selected from compounds of Formula (I):

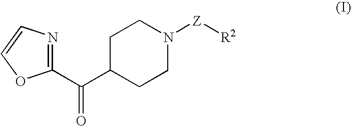

wherein:
hZ is —C(O)(CH₂)ₙ—, —SO₂—, or —CH(R^{f})—;
   where n is 0, 1, or 2; and
   R^{f} is H or C₁₋₄alkyl; and
R² is:
(a) a phenyl group, unsubstituted or substituted with one, two, or three R^{a} moieties;
   where each R^{a} moiety is: independently selected from the group consisting of C₁₋₇alkyl, —C₃₋₇cycloalkyl, —C₂₋₇alkenyl, —OH, —OC₁₋₇alkyl, —OC₃₋₇cycloalkyl, phenyl unsubstituted or substituted with R^{b}, phenoxy unsubstituted or substituted with R^{b}, halo, —CF₃, —OCF₃, —SC₁₋₄alkyl, —SO₂C₁₋₄alkyl, —SOC₁₋₄alkyl, —CN, —NO₂, —CO₂H, —CO₂C₁₋₄alkyl, —SO₂NR^{c}R^{d}, —NR^{c}SO₂R^{d}, —C(O)NR^{c}R^{d}, —NR^{c}C(O)R^{d}, and —N(R^{c})R^{d}; or two adjacent R^{a} moieties together form —O(CH₂)₁₋₂O— or —O(CF₂)O—;
      where R^{b} is selected from the group consisting of —C₁₋₄alkyl, —OC₁₋₄alkyl, halo, —CN, —OH, —CF₃, —OCF₃, and —NO₂; and
   where R^{c} and R^{d} are each independently —H or —C₁₋₇alkyl;
(b) a five- or six-membered monocyclic heteroaryl group, unsubstituted or substituted with one or two R^{a} moieties;
(c) a naphthyl group, unsubstituted or substituted with one or two R^{e} moieties;
   where each R^{e} moiety is: independently selected from the group consisting of —OC₁₋₄alkyl, halo, —CN, —OH, —CF₃, —OCF₃, and —NO₂; or two adjacent R^{e} moieties together form —O(CH₂)₁₋₂O— or —O(CF₂)O—; or
(d) a nine- or ten-membered fused bicyclic heteroaryl group, unsubstituted or substituted with one or two R^{e} moieties;
   and pharmaceutically acceptable salts of compounds of Formula (I), pharmaceutically acceptable prodrugs of compounds of Formula (I), and pharmaceutically active metabolites of Formula (I).

In certain preferred embodiments, the compound of Formula (I) is a compound selected from those species described or exemplified in the detailed description below.

In a further general aspect, the invention relates to pharmaceutical compositions each comprising: (a) an effective amount of at least one chemical entity selected from compounds of Formula (I), pharmaceutically acceptable salts of compounds of Formula (I), pharmaceutically acceptable prodrugs of compounds of Formula (I), and pharmaceutically active metabolites of Formula (I); and (b) a pharmaceutically acceptable excipient.

In another general aspect, the invention is directed to a method of treating a subject suffering from or diagnosed with a disease, disorder, or medical condition mediated by FAAH activity, comprising administering to the subject in need of such treatment an effective amount of at least one chemical entity selected from compounds of Formula (I), pharmaceutically acceptable salts of compounds of Formula (I), pharmaceutically acceptable prodrugs of compounds of Formula (I), and pharmaceutically active metabolites of compounds of Formula (I).

In certain preferred embodiments of the inventive method, the disease, disorder, or medical condition is selected from: anxiety, depression, pain, sleep disorders, eating disorders, inflammation, multiple sclerosis and other movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, symptoms of drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, auto-immune diabetes, intractable pruritis, and neuroinflammation.

Additional embodiments, features, and advantages of the invention will be apparent from the following detailed description and through practice of the invention.

### DETAILED DESCRIPTION OF INVENTION AND ITS PREFERRED EMBODIMENTS

The invention may be more fully appreciated by reference to the following description, including the following glossary of terms and the concluding examples. For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a straight- or branched-chain alkyl group having from 1 to 12 carbon atoms in the chain. Examples of alkyl groups include methyl (Me, which also may be structurally depicted by a / symbol), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “alkenyl” refers to a straight- or branched-chain alkenyl group having from 2 to 12 carbon atoms in the chain. (The double bond of the alkenyl group is formed by two sp² hybridized carbon atoms.) Illustrative alkenyl groups include prop-2-enyl, but-2-enyl, but-3-enyl, 2-methylprop-2-enyl, hex-2-enyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

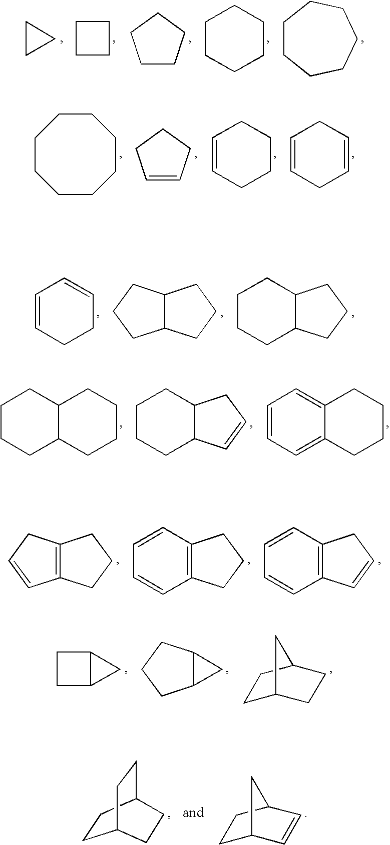

A “heterocycloalkyl” refers to a monocyclic, or fused, bridged, or Spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative examples of heterocycloalkyl groups include, in the form of properly bonded moieties:

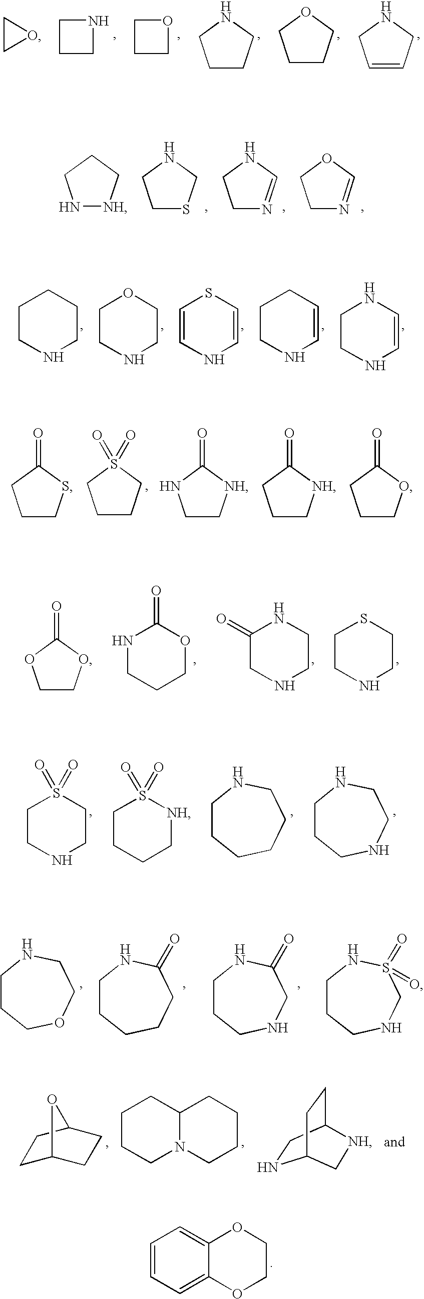

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

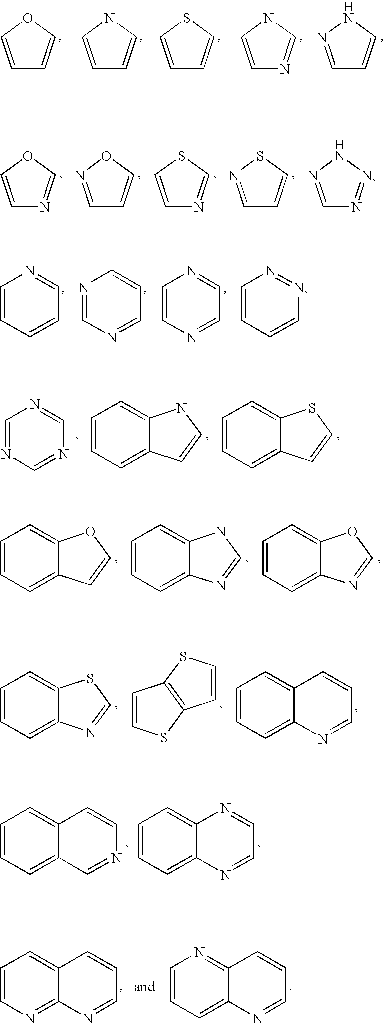

Those skilled in the art will recognize that the species of cycloalkyl, heterocycloalkyl, and heteroaryi groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo. .

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ₁₅N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula.

In preferred embodiments of Formula (I), Z is —C(O)—, —SO₂—, or —CH₂—. In other preferred embodiments, Z is —CH₂—.

In preferred embodiments, n is 2.

In preferred embodiments, R^{f} is H or CH₃.

In preferred embodiments, R² is a phenyl, pyrrolyl, furanyl, thiophenyl, pyrazolyl, imidazolyl, oxazolyl, isoxazolyl, thiazolyl, oxadiazolyl, thiadiazolyl, pyridyl, pyridazinyl, pyrimidinyl, or pyrazinyl group, unsubstituted or substituted with one, two, or three of the R^{e} moieties. In further preferred embodiments, R² is a phenyl group, unsubstituted or substituted with one, two, or three of the R^{a} moieties. In still further preferred embodiments, R² is a naphthyl, benzofuranyl, benzothiophenyl, indolyl, benzoimidazolyl, benzoxazolyl, benzothiazolyl, quinolinyl, isoquinolinyl, or naphthyridinyl group, unsubstituted or substituted with one or two of the R^{e} moieties. In other preferred embodiments, R² is a naphthyl, benzofuranyl, benzothiophenyl, indolyl, benzoimidazolyl, quinolinyl, or naphthyridinyl group, unsubstituted or substituted with one or two of the R^{e} moieties. In still other preferred embodiments, R² is phenyl, 2-methylphenyl, 4-methylphenyl, 3-methylphenyl, 4-ethylphenyl, 4-isopropylphenyl, 4-isobutylphenyl, 4-t-butylphenyl, 4-cyclohexylphenyl, 2-methoxyphenyl, 3-methoxyphenyl, 4-methoxyphenyl, 3-ethoxyphenyl, 4-ethoxyphenyl, 3-isopropoxyphenyl, 4-isopropoxyphenyl, 3-isobutyoxphenyl, 4-isobutoxyphenyl, 4-t-butoxyphenyl, 3-cyclohexyloxyphenyl, 4-cyclohexyloxyphenyl, 3-biphenyl, 4-biphenyl, 3-phenoxyphenyl, 4-phenoxyphenyl, 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 3-trifluoromethylphenyl, 4-trifluoromethylphenyl, 3-trifluoromethoxyphenyl, 4-trifluoromethoxyphenyl, 4-dimethylaminophenyl, 4-diethylaminophenyl, 2,3-dimethylphenyl, 3,4-dimethoxyphenyl, 2,3-difluorophenyl, 2,4-difluorophenyl, 2,5-difluorophenyl, 3,4-difluorophenyl, 3,4-dichlorophenyl, 3,4-dibromophenyl, 4-bromo-2-fluorophenyl, 3-chloro-4-fluorophenyl, 2,4,6-trifluorophenyl, 2,3,5-trifluorophenyl, 4-bromo-2-methanesulfanylphenyl, 4-bromo-3-nitrophenyl, benzo[1,3]dioxolyl, 2,2-difluoro-benzo[1,3]dioxo1-5-yl, 2-furanyl, 3-methyl-isoxazol-5-yl, pyridin-2-yl, pyridin-3-yl, pyridin-4-yl, 6-methyl-pyridin-2-yl, 6-bromo-pyridin-2-yl, 6-methoxy-pyridin-3-yl, 6-chloro-pyridin-3-yl, 5-bromo-pyridin-3-yl, 6-bromo-pyridin-3-yl, 6-phenoxy-pyridin-3-yl, 6-p-tolyloxy-pyridin-3-yl, 6-(3-methoxy-phenyl)-pyridin-3-yl, 6-(3-cyanophenyl)-pyridin-3-yl, napthalen-1-yl, naphthalen-2-yl, 1-hydroxy-naphthalen-2-yl, 6-methoxy-naphthalen-2-yl, 1-methyl-1H-indol-2-yl, 1H-indol-5-yl, 1-methyl-1H-indol-5-yl, 1H-indol-6-yl, 1-methyl-1H-indol-6-yl, benzofuran-2-yl, benzo[b]thiophen-2-yl, 1-methyl-1H-benzoimidazol-2-yl, 2-quinollnyl, 3-quinolinyl, 4-quinolinyl, 3-chloro-quinolin-2-yl, 6-chloro-quinolin-2-yl, 7-chloro-quinolin-2-yl, 8-chloro-quinolin-2-yl, 8-hydroxy-quinolin-2-yl, 2-chloro-quinolin-3-yl, 2-dimethylamino-quinolin-3-yl, 2-chloro-6-methyl-quinolin-3-yl, 2-chloro-8-methyl-quinolin-3-yl, 2-chloro-6-methoxy-quinolin-3-yl, 2-chloro-7-methoxy-quinolin-3-yl, 2-chloro-7-methyl-quinolin-3-yl, 2,7-dichloro-quinolin-3-yl, 6-chloro-[1,3]dioxolo[4,5-g]quinolin-7-yl, [1,8]naphthyridin-2-yl, or quinoxalin-2-yl. In further preferred embodiments, R² is benzo[1,3]dioxolyl or 2,2-difluoro-benzo[1,3]dioxol-5-yl. In still further preferred embodiments, R² is a phenyl group substituted with one or two R^{a} moieties, where each R^{a} moiety is independently selected from halo.

In preferred embodiments, each R^{a} moiety is: independently selected from the group consisting of methyl, ethyl, propyl, isopropyl, 2-methylpropyl, cyclopropyl, cyclobutyl, cyclopentyl, cyclohexyl, —OH, methoxy, ethoxy, isopropoxy, isobutoxy, cyclopentyloxy, cyclohexyloxy, phenyl unsubstituted or substituted with R^{b}, phenoxy unsubstituted or substituted with R^{b}, fluoro, chloro, bromo, —CF₃, —OCF₃, methanesulfanyl, methanesulfonyl, —CN, —NO₂, methoxycarbonyl, ethoxycarbonyl, —CO₂H, acetyl, —SO₂NR^{c}R^{d}, —NR^{c}SO₂R^{d}, —C(O)NR^{c}R^{d}, —NR^{c}C(O)R^{d}, and —N(R^{c})R^{d}; or two adjacent R^{a} moieties together form —O(CH₂)₁₋₂O—or —O(CF₂)O—.

In preferred embodiments, R^{b} is selected from the group consisting of methyl, ethyl, isopropy, methoxy, ethoxy, fluoro, chloro, bromo, —CN, —OH, —CF₃, —OCF₃, and —NO₂.

In preferred embodiments, R^{c} and R^{d} are each independently H, methyl, ethyl, or isopropyl.

In preferred embodiments, each R^{e} moiety is: independently selected from the group consisting of methyl, ethyl, propyl, isopropyl, methoxy, ethoxy, propoxy, isopropoxy, fluoro, chloro, bromo, —CN, —OH, —CF₃, —OCF₃, and —NO₂; or two adjacent R^{e} moieties together form —O(CH₂)₁₋₂O— or —O(CF₂)O—.

The invention includes also pharmaceutically acceptable salts of the compounds represented by Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenyipropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, omithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl)esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I), and uses of such metabolites in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J. Med. Chem. 1997, 40, 2011-2016; Shan, et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites (collectively, “active agents”) of the present invention are useful as FAAH inhibitors in the methods of the invention. The active agents may be used in the inventive methods for the treatment or prevention of medical conditions, diseases, or disorders mediated through inhibition or modulation of FAAH, such as those described herein. Active agents according to the invention may therefore be used as an analgesic, anti-depressant, cognition enhancer, neuroprotectant, sedative, appetite stimulant, or contraceptive.

Exemplary medical conditions, diseases, and disorders include anxiety, depression, pain, sleep disorders, eating disorders, inflammation, multiple sclerosis and other movement disorders, HIV wasting syndrome, closed head injury, stroke, learning and memory disorders, Alzheimer's disease, epilepsy, Tourette's syndrome, epilepsy, Niemann-Pick disease, Parkinson's disease, Huntington's chorea, optic neuritis, autoimmune uveitis, symptoms of drug withdrawal, nausea, emesis, sexual dysfunction, post-traumatic stress disorder, or cerebral vasospasm.

Thus, the active agents may be used to treat subjects diagnosed with or suffering from a disease, disorder, or condition mediated through FAAH activity. The term “treat” or “treating” as used herein is intended to refer to administration of an agent or composition of the invention to a subject for the purpose of effecting a therapeutic or prophylactic benefit through modulation of FAAH activity. Treating includes reversing, ameliorating, alleviating, inhibiting the progress of, lessening the severity of, or preventing a disease, disorder, or condition, or one or more symptoms of such disease, disorder or condition mediated through modulation of FAAH activity. The term “subject” refers to a mammalian patient in need of such treatment, such as a human. “Modulators” include both inhibitors and activators, where “inhibitors” refer to compounds that decrease, prevent, inactivate, desensitize or down-regulate FAAH expression or activity, and “activators” are compounds that increase, activate, facilitate, sensitize, or up-regulate FAAH expression or activity.

Accordingly, the invention relates to methods of using the active agents described herein to treat subjects diagnosed with or suffering from a disease, disorder, or condition mediated through FAAH activity, such as: anxiety, pain, sleep disorders, eating disorders, inflammation, or movement disorders (e.g., multiple sclerosis).

Symptoms or disease states are intended to be included within the scope of “medical conditions, disorders, or diseases.” For example, pain may be associated with various diseases, disorders, or conditions, and may include various etiologies. Illustrative types of pain treatable with a FAAH-modulating agent according to the invention include cancer pain, postoperative pain, GI tract pain, spinal cord injury pain, visceral hyperalgesia, thalamic pain, headache (including stress headache and migraine), low back pain, neck pain, musculoskeletal pain, peripheral neuropathic pain, central neuropathic pain, neurogenerative disorder related pain, and menstrual pain. HIV wasting syndrome includes associated symptoms such as appetite loss and nausea. Parkinson's disease includes, for example, levodopa-induced dyskinesia. Treatment of multiple sclerosis may include treatment of symptoms such as spasticity, neurogenic pain, central pain, or bladder dysfunction. Symptoms of drug withdrawal may be caused by, for example, addiction to opiates or nicotine. Nausea or emesis may be due to chemotherapy, postoperative, or opioid related causes. Treatment of sexual dysfunction may include improving libido or delaying ejaculation. Treatment of cancer may include treatment of glioma. Sleep disorders include, for example, sleep apnea, insomnia, and disorders calling for treatment with an agent having a sedative or narcotic-type effect. Eating disorders include, for example, anorexia or appetite loss associated with a disease such as cancer or HIV infection/AIDS.

In treatment methods according to the invention, an effective amount of at least one active agent according to the invention is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.001 to about 200 mg of active agent per kg of subject's body weight per day, preferably about 0.05 to 100 mg/kg/day, or about 1 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 2.5 g/day. Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the active agents of the invention may be used in combination with additional active ingredients in the treatment of the above conditions. The additional active ingredients may be coadministered separately with an active agent of Formula (I) or included with such an agent in a pharmaceutical composition according to the invention. In an exemplary embodiment, additional active ingredients are those that are known or discovered to be effective in the treatment of conditions, disorders, or diseases mediated by FAAH activity, such as another FAAH modulator or a compound active against another target associated with the particular condition, disorder, or disease. The combination may serve to increase efficacy (e.g., by including in the combination a compound potentiating the potency or effectiveness of an active agent according to the invention), decrease one or more side effects, or decrease the required dose of the active agent according to the invention. In one illustrative embodiment, a composition according to the invention may contain one or more additional active ingredients selected from opioids, NSAIDs (e.g., ibuprofen, cyclooxygenase-2 (COX-2) inhibitors, and naproxen), gabapentin, pregabalin, tramadol, acetaminophen, and aspirin.

The active agents of the invention are used, alone or in combination with one or more additional active ingredients, to formulate pharmaceutical compositions of the invention. A pharmaceutical composition of the invention comprises: (a) an effective amount of at least one active agent in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active Agents of the invention can be provided in the form of tablets or capsules, or as a solution, emulsion, or suspension. To prepare the oral compositions, the active agents may be formulated to yield a dosage of, e.g., from about 0.05 to about 50 mg/kg daily, or from about 0.05 to about 20 mg/kg daily, or from about 0.1 to about 10 mg/kg daily.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

Exemplary chemical entities useful in methods of the invention will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I).

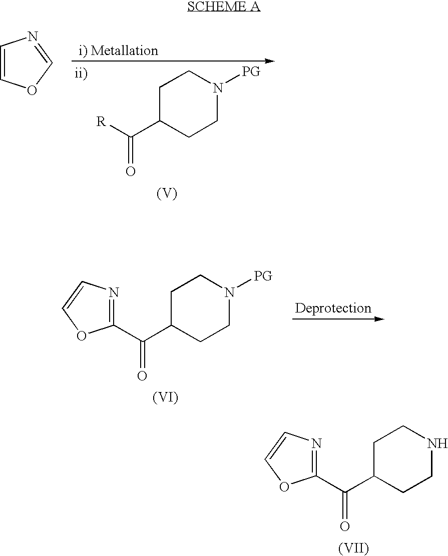

Amino-ketones (VII) are useful in the preparation of compounds of Formula (I). To access amino-ketones (VII), oxazole is metallated and coupled with reagents (V), where R is Cl or —N(OMe)(Me) and PG is a suitable nitrogen protecting group such as a benzyl or t-butylcarbamate (Boc). Reagents (V) may be selected from commercially available materials or prepared by suitably applying synthetic methods known in the art. Metallation of oxazole may be accomplished according to various procedures. In one embodiment,.oxazole is lithiated at the 2-position by treatment with n-BuLi or sec-BuLi, at temperatures of about −78° C., in a solvent such as THF. Direct coupling of a lithiated oxazole with reagents (V) will generate ketones (VI) (Boger et al., J. Med. Chem. 2005, 48(6), 1849-1856). Alternatively, the 2-lithio-oxazoles are transmetallated in situ to their corresponding zinc reagents by treatment with ZnCl₂. Reaction solutions may be warmed to about 0° C. Subsequent in situ treatment of the zinc reagents with a Copper(I) species such as CuI gives metallated oxazoles that may be coupled with compounds of formula (V) to give ketones (VI). See: Boger, D. et al. PNAS 2000, 97(10), 5044-5049. Deprotection of ketones (VI) is accomplished by suitably applying deprotection methods known in the art to provide amino-ketones (VII). In a preferred embodiment, PG is a Boc group, and is removed by treatment with HCl in dioxane or with trifluoroacetic acid (TFA).

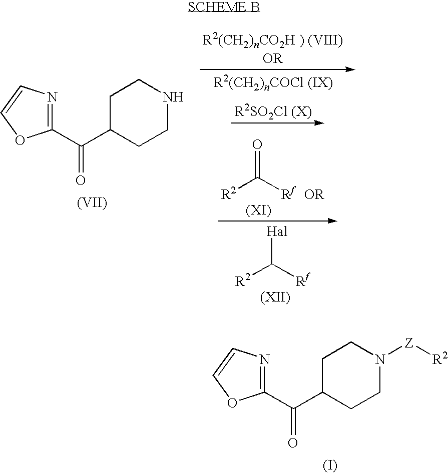

Compounds of Formula (I) where Z is —C(O)(CH₂)ₙ— are available by reaction of piperidines (VII) with: 1) a suitably substituted acid (VIII) in the presence of suitable amide coupling agents, such as CDI, EDC/HOBt, or HATU, in a solvent such as THF, DMF, or acetonitrile; or 2) a suitably substituted acid chloride (IX), in the presence of an amine base such as Et₃N or iPr₂NEt, in a solvent such as DCM or DCE. Compounds of Formula (I) where Z is —SO₂— are available by reaction of piperidines (VII) with a suitable sulfonyl chloride (X), in the presence of a suitable amine base such as Et₃N or iPr₂NEt, in a solvent such as DCM or DCE. Compounds of Formula (I) where Z is —CH(R^{f})— are available by: 1) reductive amination with a suitable aldehyde or ketone (XI), in the presence of a reducing agent such as Na(CN)BH₃ or Na(OAc)₃BH, in a solvent such as DCM, MeOH, or EtOH; or 2) alkylation with a suitable alkyl halide (XII), where Hal is Br, Cl, or I, in the presence of a base such as K₂CO₃, Na₂CO₃, or Cs₂CO₃, and optional additives such as NaI or KI, in a polar solvent such as acetonitrile or DMF.

Compounds of Formula (I) may be converted to their corresponding salts using methods described in the art. For example, amines of Formula (I) may be treated with trifluoroacetic acid, HCl, or citric acid in a solvent such as Et₂O, CH₂Cl₂, THF, or MeOH to provide the corresponding salt forms.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following specific examples are provided to further illustrate the invention and various preferred embodiments.

### Examples

### Chemistry:

Where solutions or mixtures are concentrated, they are typically concentrated under reduced pressure using a rotary evaporator.

Normal phase flash column chromatography (FCC) was performed on silica gel columns using EtOAc/hexanes as eluent, unless otherwise indicated.

Preparative Reversed-Phase high performance liquid chromatography (HPLC) was performed using a Gilson® instrument with a YMC-Pack ODS-A, 5 μm, 75×30 mm column, a flow rate of 25 mL/min, detection at 220 and 254 nm, with a 15% to 99% acetonitrile/water/0.05% TFA gradient, unless otherwise indicated.

Analytical Reversed-Phase HPLC was performed using 1) a Hewlett Packard Series 1100 instrument with an Agilent ZORBAX® Bonus RP, 5 μm, 4.6×250 mm column, a flow rate of 1 mL/min, detection at 220 and 254 nm, with a 1% to 99% acetonitrile/water/0.05% TFA gradient; or 2) a Hewlett Packard HPLC instrument with an Agilent ZORBAX® Eclipse XDB-C8, 5 μm, 4.6×150 mm column, a flow rate of 1 mL/min, detection at 220 and 254 nm, with a 1% to 99% acetonitrile/water/0.05% TFA gradient, unless otherwise indicated.

Thin-layer chromatography was performed using Merck silica gel 60 F₂₅₄ 2.5 cm×7.5 cm 250 μm or 5.0 cm×10.0 cm 250 μm pre-coated silica gel plates. Preparative thin-layer chromatography was performed using EM Science silica gel 60 F₂₅₄ 20 cm×20 cm 0.5 mm pre-coated plates with a 20 cm×4 cm concentrating zone.

In obtaining the characterization data described in the examples below, the following analytical protocols were followed unless otherwise indicated.

Mass spectra were obtained on an Agilent series 1100 MSD using electrospray ionization (ESI) in either positive or negative modes as indicated. Calculated mass corresponds to the exact mass.

NMR spectra were obtained on either a Bruker model DPX400 (400 MHz), DPX500 (500 MHz), DRX600 (600 MHz) spectrometer. The format of the ¹H NMR data below is: chemical shift in ppm down field of the tetramethylsilane reference (multiplicity, coupling constant J in Hz, integration).

Where a potential chiral center is designated with a solid bond (not bold or hashed), the structure is meant to refer to a racemic mixture.

Chemical names were generated using ChemDraw Ultra 6.0.2 (CambridgeSoft Corp., Cambridge, Mass.).

### Intermediate 1: 4-Chlorocarbonyl-piperidine-1-carboxylic acid tert-butyl ester

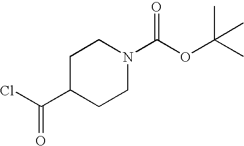

To a solution of piperidine-1,4-dicarboxylic acid mono-tert-butyl ester (5.0 g) in diethyl ether (Et₂O; 100 mL) was added pyridine (0.90 mL) followed by SOCl₂ (1.7 mL) dropwise. The mixture was stirred for 2 h and then filtered. The filtrate was concentrated and dried under vacuum to give a colorless oil (5.5 g).

### Intermediate 2: 4-(Oxazole-2-carbonyl)-piperidine-1-carboxylic acid tert-butyl ester

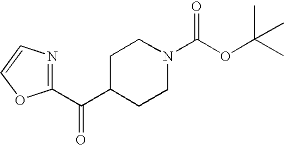

A −78° C. solution of oxazole (1.6 mL) in THF (100 mL) was treated with n-BuLi (1.6 M in hexanes; 16.4 mL). The resulting mixture was treated with ZnCl₂ (1.0 M in Et₂O; 26.2 mL), cooled to 0 C, and stirred for 45 min. Copper(I) iodide (5.0 g) was added, and after 10 min, Intermediate 1 (5.5 g) was added. The resulting mixture was allowed to warm to room temperature (rt) overnight, then was diluted with EtOAc (50 mL) and washed with 50% aq. NH₃ (40 mL). The aqueous layer was extracted with EtOAc (2×50 mL). The combined organic layers were washed with water (2×40 mL) and saturated aqueous (satd. aq.) NaCl (40 mL), and dried (MgSO₄). Concentration and purification by FCC gave the title compound as a colorless oil (2.06 g). ¹H NMR (CDCl₃): 7.84 (d, J=0.8 Hz, 1H), 7.34 (d, J=0.5 Hz, 1H), 4.18 (bs, 2H), 3.62-3.54 (m, 1H), 2.95-2.83 (m, 2H), 2.02-1.90 (m, 2H), 1.76-1.63 (m, 2H), 1.47 (s, 9H).

### Intermediate 3: Oxazol-2-yl-piperidin-4-yl-methanone hydrochloride

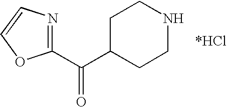

To a solution of Intermediate 2 (2.8 g) in dioxane (14 mL) was added HCl (4 N in dioxane, 28 mL). The resulting mixture was stirred for 18 h and concentrated to give the title compound as a white solid (2.0 g). ¹H NMR (CD₃OD): 8.17 (d, J=0.8 Hz, 1H), 7.46 (d, J=0.8 Hz, 1H), 3.80-3.72 (m, 1H), 3.50-3.45 (m, 2H), 3.20-3.13 (m, 2H), 2.28-2.23 (m, 2H), 1.98-1.87 (m, 2H). MS: calcd for C₉H₁₂N₂O₂, 180.1; m/z found, 181.1 [M+H]⁺.

### Intermediate 4: 1-Methyl-1H-indole-5-carbaldehyde

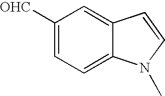

A solution of indole-5-carbaldehyde (0.5 g) in dimethyl carbonate (5 mL) was treated with 1,4-diaza-bicyclo[2.2.2]octane (38 mg). After 5 h at 90° C., the mixture was diluted with water (10 mL) and extracted with EtOAc (3×10 mL). The combined organic extracts were washed with satd. aq. NaCl (1×20 mL), dried (MgSO₄), and concentrated. Purification by FCC gave the title compound as a white solid (46%). ¹H NMR (CDCl₃): 10.08 (s, 1H), 7.92-7.90 (m, 1H), 7.72 (d, J=8.4 Hz, 1H), 7.66-7.62 (m, 1H), 7.29 (d, J=3.0 Hz, 1H), 6.58-6.55 (m, 1H), 3.90 (s, 3H).

### Intermediate 5: 1-Methyl-1H-indole-6-carbaldehyde

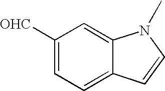

The title compound was prepared in analogy with Intermediate 4, using 1H-indole-6-carbaldehyde. ¹H NMR (CDCl₃): 10.03 (s, 1H), 8.16 (d, J=1.5 Hz, 1H), 7.82-7.78 (m, 1H), 7.41 (d, J=8.6 Hz, 1H), 7.15 (d, J=3.0 Hz, 1H), 6.67-6.65 (m, 1H), 3.85 (s, 3H).

### Intermediate 6: 3-Cyclohexyloxy-benzaldehyde

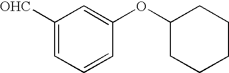

A solution of 3-hydroxybenzaldehyde (5.0 g), cyclohexanol (4.1 g), and triphenylphosphine (16.16 g) in THF (205 mL), was treated dropwise with diethyl azodicarboxylate (DEAD; 11.19 mL). The resulting mixture was heated at reflux for 24 h, cooled to rt and diluted with Et₂O (100 mL). The mixture was washed with water (2×100 mL), 0.4 N NaOH (2×50 mL), water (100 mL), and satd. aq. NaCl (50 mL). The organic phase was dried (Na₂SO₄) and concentrated, and the residue was purified by FCC to give the title compound as a yellow oil (1.75 g). ¹H NMR (CDCl₃): 9.96 (s, 1H), 7.44-7.38 (m, 3H), 7.17-7.15 (m, 1H), 4.36-4.31 (m, 1H), 1.99-1.97 (m, 2H), 1.81-1.79 (m, 2H), 1.57-1.49 (m, 3H), 1.43-1.30 (m, 3H).

### Intermediate 7: 4-Cyclohexyloxy-benzaldehyde

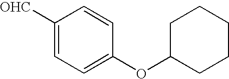

A mixture of 4-hydroxybenzaldehyde (10.0 g), cyclohexyl bromide (48.1 mL), and K₂CO₃ (19.5 g) in DMF (48 mL) was heated at 90° C. for two days. After cooling, the mixture was diluted with 1:1 hexanes/EtOAc (48 mL), washed with water (2×50 mL), 2 N NaOH (3×50 mL), water (50 mL), and satd. aq. NaCl (50 mL), dried (Na₂SO₄) and concentrated, giving the title compound as an orange oil (3.57 g). ¹H NMR (CDCl₃): 9.86 (s, 1H), 7.83-7.79 (m, 2H), 6.99-6.96 (m, 2H), 4.41-4.35 (m, 1H), 2.01-1.98 (m, 2H), 1.83-1.81 (m, 2H), 1.60-1.56 (m, 3H), 1.42-1.37 (m, 3H).

### Intermediate 8: 3-Isopropoxy-benzaldehyde

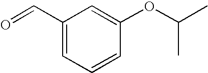

A solution of 3-hydroxybenzaldehyde (4.5 g) and 2-iodopropane (3.72 mL) in 2-propanol (40 mL) was treated with K₂CO₃ (16.09 g). After 8 h at reflux, water (40 mL) was added and the mixture was extracted with Et₂O (3×25mL). The combined organic layers were washed with water (25 mL), 2 M NaOH (25 mL), water (4×25 mL), and satd. aq. NaCl (25 mL). The organic phase was dried (Na₂SO₄), and concentrated to give the title compound as a pale orange oil (3.31 g). ¹H NMR (CDCl₃): 9.96 (s, 1H), 7.45-7.41 (m, 21-I), 7.38-7.37 (m, 1H), 7.17-7.13 (m, 1H), 4.68-4.59 (septet, 1H, J=6.1 Hz), 1.37-1.35 (d, 6H, J=6.1 Hz).

### Intermediate 9: 6-Chloro-quinoline-2-carbaldehyde

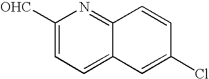

A suspension of 6-chloro-2-methyl-quinoline (355 mg) and SeO₂ (233 mg) in 1,4-dioxane (3 mL) was heated to 80° C. for 16 h. The resulting black mixture was filtered through diatomaceous earth. Concentration of the filtrate gave the title compound as a yellow powder (324 mg). ¹H NMR (CDCl₃): 10.21 (d, J=0.8 Hz, 1H), 8.26-8.18 (m, 2H), 8.06 (d, J=8.6 Hz, 1H), 7.91 (d, J=2.3 Hz, 1H), 7.79-7.75 (m, 1H).

### Intermediate 10: 8-Chloro-quinoline-2-carbaldehyde

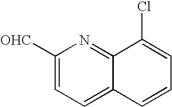

The title compound was prepared in analogy with Intermediate 9, using 8-chloro-2-methyl-quinoline. ¹H NMR (CDCl₃): 10.32 (d, J=0.8 Hz, 1H), 8.36 (d, J=8.3 Hz, 1H), 8.11 (d, J=8.3 Hz, 1H), 7.97-7.94 (m, 1H), 7.87-7.84 (m, 1H), 7.65-7.60 (m, 1H).

### Intermediate 11

### 7-Chloro-quinoline-2-carbaldehyde

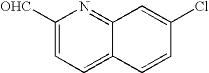

The title compound was prepared in analogy with Intermediate 9, using 7-chloro-2-methyl-quinoline. ¹H NMR (CDCl₃): 10.21 (d, J=0.8 Hz, 1H), 8.31 (d, J=8.6 Hz, 1H), 8.26 (d, J=2.0 Hz, 1H), 8.03 (d, J=8.3 H, 1H), 7.86 (d, J=8.6 Hz, 1H), 7.67-7.64 (m, 1H).

### Example 1

### [1-(Naphthalene-2-sulfonyl)-piperidin-4-yl]-oxazol-2-yl-methanone

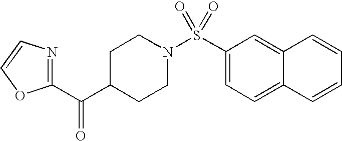

To a suspension of Intermediate 3 (54 mg) in DCM (3 mL) was added 2-naphthalenesulfonyl chloride (63 mg) followed by Et₃N (0.074 mL). After 1 h, the resulting mixture was purified by FCC (2 M NH₃ in MeOH/DCM) to give the title compound as a white solid (80 mg). ¹H NMR (CDCl₃): 8.35 (d, J=1.5 Hz, 1H), 7.99 (d, J=8.6 Hz, 2H), 7.95-7.93 (m, 1H), 7.80 (d, J=1.0 Hz, 1H), 7.78-7.76 (m, 1H), 7.70-7.61 (m, 2H), 7.27 (s, 1H), 3.92-3.87 (m, 2H), 3.35-3.28 (m, 1H), 2.62-2.55 (m, 2H), 2.09-2.05 (m, 2H), 1.96-1.86 (m, 2H). MS: calcd for C₁₉H₁₈N₂O₄S, 370.1; m/z found, 371.1 [M+H]⁺.

The compounds in Examples 2-6 were prepared using methods analogous to those described in Example 1.

### Example 2

### [1-(4-Methoxy-benzenesulfonyl)-piperidin-4-yl]-oxazol-2-yl-methanone

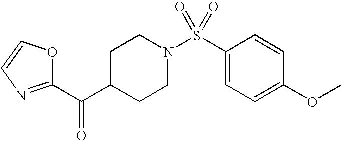

¹H NMR (CDCl₃): 7.82 (d, J=0.5 Hz, 1H), 7.74-7.69 (m, 2H), 7.29 (d, J=0.5 Hz, 1H), 7.03-6.98 (m, 2H), 3.89 (s, 3H), 3.82-3.76 (m, 2H), 3.38-3.30 (m, 1H), 2.54-2.46 (m, 2H), 2.10-2.02 (m, 2H), 1.95-1.84 (m, 2H). MS: calcd for C₁₆H₁₈N₂O₅S, 350.1; m/z found, 351.0 [M+H]⁺.

### Example 3

### [1-(3-Methoxy-benzenesulfonyl)-piperidin-4-yl]-oxazol-2-yl-methanone

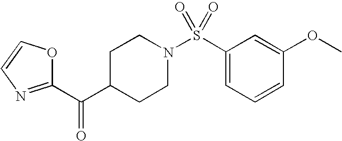

¹H NMR (CDCl₃): 7.82 (d, J=0.5 Hz, 1H), 7.47-7.43 (m, 1H), 7.37-7.33 (m, 1H), 7.30 (d, J=0.5 Hz, 1H), 7.28-7.27 (m, 1H), 7.16-7.12 (m, 1H), 3.87 (s, 3H), 3.85-3.78 (m, 2H), 3.40-3.31 (m, 1H), 2.59-2.50 (m, 2H), 2.11-2.03 (m, 2H), 1.96-1.84 (m, 2H). MS: calcd for C₁₆H₁ₑN₂O₅S, 350.1; m/z found, 351.0 [M+H]⁺.

### Example 4

### [1-(4-Chloro-benzenesulfonyl)-piperidin-4-yl]-oxazol-2-yl-methanone

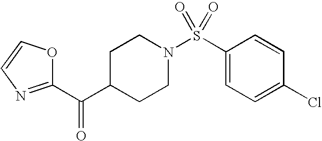

¹H NMR (CDCl₃): 7.82 (d, J=0.8 Hz, 1H), 7.74-7.70 (m, 2H), 7.55-7.51 (m, 2H), 7.30 (d, J=0.5 Hz, 1H), 3.83-3.76 (m, 2H), 3.41-3.32 (m, 1H), 2.59-2.51 (m, 2H), 2.13-2.04 (m, 2H), 1.96-1.84 (m, 2H) MS: calcd for C₁₅H₁₅ClN₂O₄S, 354.0; m/z found, 355.0 [M+H]⁺.

### Example 5:

### [1-(3-Chloro-benzenesulfonyl)-Diperidin-4-yl]-oxazol-2-yl-methanone

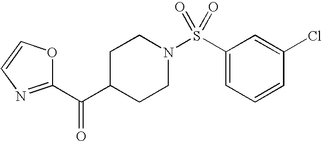

¹H NMR (CDCl₃): 7.82 (d, J=0.8 Hz, 1H), 7.78-7.76 (m, 1H), 7.68-7.65 (m, 1H), 7.61-7.58 (m, 1H), 7.52-7.47 (m, 1H), 7.30 (d, J=0.5 Hz, 1H), 3.86-3.78 (m, 2H), 3.42-3.34 (m, 1H), 2.62-2.53 (m, 2H), 2.13-2.04 (m, 2H), 1.97-1.85 (m, 2H). MS: calcd for C₁₅H₁₅ClN₂O₄S, 354.0; m/z found, 355.0 [M+H]⁺.

### Example 6

### [1-(2-Chloro-benzenesulfonyl)-piperidin-4-yl]-oxazol-2-yl-methanone

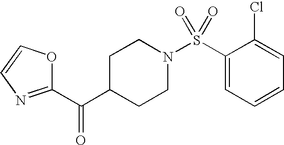

¹H NMR (CDCl₃): 8.09-8.06 (m, 1H), 7.84-7.83 (d, J=0.8 Hz, 1H), 7.56-7.47 (m, 2H), 7.43-7.38 (m, 1H), 7.33 (d, J=0.5 Hz, 1H), 3.96-3.86 (m, 2H), 3.56-3.46 (m, 1H), 3.04-2.92 (m, 2H), 2.10-2.01 (m, 2H), 1.91-1.80 (m, 2H). MS: calcd for C₁₅H₁₅ClN₂O₄S, 354.0; m/z found, 355.0 [M+H]⁺.

### Example 7

### [1-(3-Methoxy-benzoyl)-piperidin-4-yl]-oxazol-2-yl-methanone

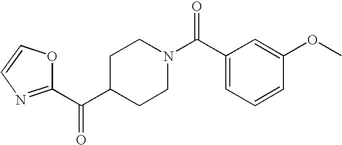

To a suspension of oxazol-2-yl-piperidin-4-yl-methanone (65 mg) in DCM (4 mL) was added Et₃N (89 μL). After 15 min at rt, the suspension was treated with meta-anisoyl chloride (46.4 μL). After 1 h at rt, the mixture was purified directly by FCC to give the title compound (85.6 mg). ¹H NMR (CDCl₃): 7.86 (d, J=0.8 Hz, 1H), 7.36-7.29 (m, 2H), 6.99-6.94 (m, 3H), 4.73 (bs, 1H), 3.94-3.80 (m, 4H), 3.77-3.67 (m, 1H), 3.24-2.94 (m, 2H), 2.25-1.70 (m, 4H). MS: calcd for C₁₇H₁₈N₂O₄, 314.1; m/z found, 315.1 [M+H]⁺.

The compounds in Examples 8-14 were prepared using methods analogous to those described in Example 7.

### Example 8

### [1-(2-Methoxy-benzoyl)-piperidin-4-yl]-oxazol-2-yl-methanone

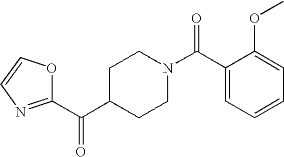

¹H NMR (CDCl₃): 7.85 (s, 1H), 7.39-7.32 (m, 2H), 7.26-7.21 (m, 1H), 7.03-6.96 (m, 1H), 6.99 (d, J=27.3 Hz, 1H), 4.95-4.69 (m, 1H), 3.84 (d, J=4.0 Hz, 3H), 3.75-3.55 (m, 2H), 3.25-2.89 (m, 2H), 2.22-2.09 (m, 1H), 1.91-1.57 (m, 3H). MS: calcd for C₁₇H₁₈N₂O₄, 314.1; m/z found, 315.1 [M+H]⁺.

### Example 9

### [1-(4-Methoxy-benzoyl)-piperidin-4-yl]-oxazol-2-yl-methanone

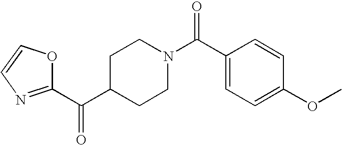

¹H NMR (CDCl₃): 7.85 (d, J=0.8 Hz, 1H), 7.42-7.37 (m, 2H), 7.35 (d, J=0.8 Hz, 1H), 6.94-6.89 (m, 2H), 4.61 (bs, 2H), 3.84 (s, 3H), 3.77-3.67 (m, 1H), 3.08 (bs, 2H), 2.02 (bs, 2H), 1.88-1.73 (m, 2H). MS: calcd for C₁₇H₁₈N₂O₄, 314.1; m/z found, 315.1 [M+H]⁺.

### Example 10

### [1-(Naphthalene-2-carbonyl)-piperidin-4-yl]-oxazol-2-yl-methanone

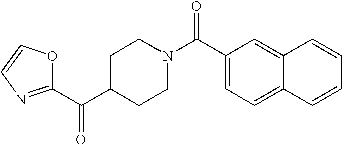

¹H NMR (CDCl₃): 7.95-7.83 (m, 5H), 7.60-7.45 (m, 3H), 7.36 (d, J=0.8 Hz, 1H), 4.80 (bs, 1H), 3.94 (bs, 1H), 3.82-3.64 (m, 1H), 3.14 (bs, 2H), 2.18 (bs, 1H), 1.98-1.76 (m, 3H). MS: calcd for C₂₀H₁₈N₂O₃, 334.13; m/z found, 335.1 [M+H]⁺.

### Example 11

### [1-(4-Chloro-benzoyl)-piperidin-4-yl]-oxazol-2-yl-methanone

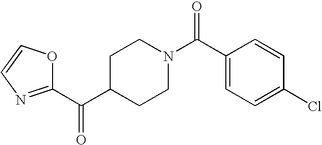

¹H NMR (CDCl₃): 7.86 (d, J=0.8 Hz, 1H), 7.42-7.35 (m, 5H), 4.71 (bs, 1H), 3.96-3.62 (m, 2H), 3.29-2.94 (m, 2H), 2.27-1.70 (m, 4H). MS: calcd for C₁₆H₁₅ClN₂O₃, 318.1; m/z found, 319.0 [M+H]⁺.

### Example 12

### [1-(3-Chloro-benzoyl)-piperidin-4-yl]-oxazol-2-yl-methanone

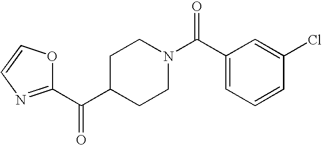

¹H NMR (CDCl₃): 7.86 (d, J=0.8 Hz, 1H), 7.42-7.28 (m, 5H), 4.71 (bs, 1H), 3.89-3.68 (m, 2H), 3.29-2.93 (m, 2H), 2.27-1.72 (m, 4H). MS: calcd for C₁₆H₁₅ClN₂O₃, 318.1; m/z found, 319.0 [M+H]⁺.

### Example 13

### [1-(2-Chloro-benzoyl)-piperidin-4-yl]-oxazol-2-yl-methanone

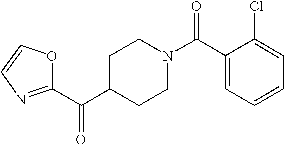

¹H NMR (CDCl₃): 7.85 (s, 1H), 7.44-7.38 (m, 1H), 7.37-7.25 (m, 4H), 4.87-4.73 (m, 1H), 3.77-3.62 (m, 1H), 3.60-3.50 (m, 1H), 3.29-2.98 (m, 2H), 2.34-2.13 (m, 1H), 2.03-1.54 (m, 3H); MS calcd for C₁₆H₁₅ClN₂O₃, 318.1; m/z found, 319.0 [M+H]⁺.

### Example 14

### 1-[4-(Oxazole-2-carbonyl)-piperidin-1-yl]-3-phenyl-propan-1-one

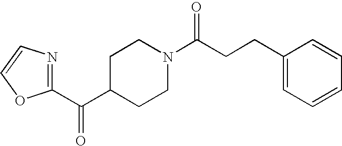

¹H NMR (CDCl₃): 7.84 (d, J=1.0 Hz, 1H), 7.34 (d, J=0.8 Hz, 1H), 7.32-7.19 (m, 5H), 4.69-4.63 (m, 1H), 3.91-3.85 (m, 1H), 3.67-3.59 (m, 1H), 3.14-3.07 (m, 1H), 3.00-2.96 (m, 2H), 2.83-2.77 (m, 1H), 2.67-2.62 (m, 2H), 2.08-2.01 (m, 1H), 1.94-1.88 (m, 1H), 1.70-1.56 (m, 2H). MS: calcd for C₁₈H₂₀N₂O₃, 312.2; m/z found, 313.3 [M+H]⁺.

### Example 15

### (1-Benzo[1,3]dioxol-5-ylmethyl-piperidin-4-yl)-oxazol-2-yl-methanone

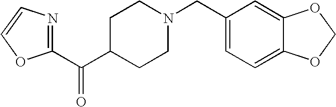

To a suspension of Intermediate 3 (65 mg) in DCM (4 mL) was added Et₃N (0.041 mL). After 30 min, the resulting mixture was treated with piperonal (50 mg) followed by NaB(OAc)₃H (89 mg). After 16 h, the resulting mixture was treated with NaOH (2 N in water, 2 mL) and loaded onto a Varian Chem Elut filter. The filter was rinsed with DCM (2×5 mL) and the combined filtrate was concentrated. Purification of the residue by FCC (2 M NH₃ in MeOH/DCM) gave the title compound as a white solid (56 mg). ¹H NMR (CDCl₃): 7.82 (d, J=0.5 Hz, 1H), 7.32 (d, J=0.8 Hz, 1H), 6.86 (s, 1H), 6.75 (d, J=0.8 Hz, 2H), 5.94 (s, 2H), 3.45-3.36 (m, 3H), 2.98-2.93 (m, 2H), 2.15-2.09 (m, 2H), 1.97-1.79 (m, 4H). MS: calcd for C₁₇H₁₆N₂O₄, 314.1; m/z found, 315.1 [M+H]⁺.

The compounds in Examples 16-89 were prepared using methods analogous to those described in Example 15.

### Example 16

### [1-(3-Isobutoxy-benzyl)-piperidin-4-yl]-oxazol-2-yl-methanone

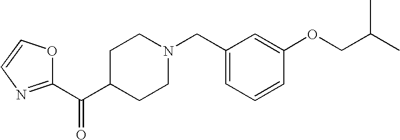

¹H NMR (CDCl₃): 7.82 (d, J=0.8 Hz, 1H), 7.33 (d, J=0.5 Hz, 1H), 7.24-7.18 (m, 1H), 6.92-6.86 (m, 2H), 6.81-6.77 (m, 1H), 3.72 (d, J=6.8 Hz, 2H), 3.50 (s, 2H), 3.46-3.36 (m, 1H), 3.01-2.94 (m, 2H), 2.18-2.02 (m, 3H), 2.00-1.78 (m, 4H), 1.03 (d, J=7.1 Hz, 6H). MS: calcd for C₂ₒH₂₆N₂O₃, 342.2; m/z found, 343.1 [M+H]⁺.

### Example 17

### [1-(4-Isobutoxy-benzyl)-piperidin-4-yl]-oxazol-2-yl-methanone

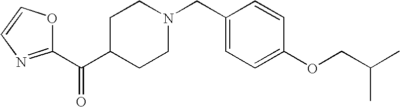

¹H NMR (CDCl3): 7.81 (d, J=0.5 Hz, 1H), 7.32 (d, J=0.5 Hz, 1H), 7.24-7.19 (m, 2H), 6.88-6.82 (m, 2H), 3.71 (d, J=6.3 Hz, 2H), 3.47 (s, 2H), 3.44-3.34 (m, 1H), 3.02-2.91 (m, 2H), 2.15-2.01 (m, 3H), 1.99-1.77 (m, 4H), 1.02 (d, J=6.6 Hz, 6H). MS: calcd for C₂₀H₂₆N₂O₃, 342.2; m/z found, 343.1 [M+H]⁺.

### Example 18

### Oxazol-2-yl-[1-(3-trifluoromethyl-benzyl)-piperidin-4-yl]-methanone

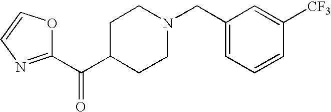

¹H NMR (CDCl₃): 7.82 (d, J=0.8 Hz, 1H), 7.59 (s, 1H), 7.56-7.49 (m, 2H), 7.46-7.40 (m, 1H), 7.33 (d, J=0.5 Hz, 1H), 3.58 (s, 2H), 3.48-3.37 (m, 1H), 2.9-2.91 (m, 2H), 2.21-2.12 (m, 2H), 2.01-1.79 (m, 4H). MS: calcd for C₁₇H₁₇F₃N₂O₂, 338.1; m/z found, 339.1 [M+H]⁺.

### Example 19

### Oxazol-2-yl-[1-(4-trifluoromethyl-benzyl)-piperidin-4-4-methanone

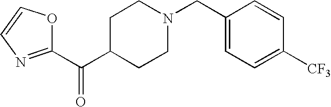

¹H NMR (CDCl₃): 7.83 (d, J=0.5 Hz, 1H), 7.57 (d, J=8.3 Hz, 2H), 7.46 (d, J=8.8 Hz, 2H), 7.33 (d, J=0.5 Hz, 1H), 3.58 (s, 2H), 3.47-3.38 (m, 1H), 2.97-2.91 (m, 2H), 2.21-2.12 (m, 2H), 2.01-1.79 (m, 4H). MS: calcd for C₁₇H₁₇F₃N₂O₂, 338.1; m/z found, 339.1 [M+H]⁺.

### Example 20

### [1-(4-Dimethylamino-benzyl)-piperidin-4-yl]-oxazol-2-yl-methanone

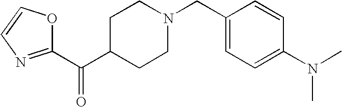

¹H NMR (CDCl₃): 7.81 (d, J=0.8 Hz, 1H), 7.32 (d, J=0.5 Hz, 1H), 7.20-7.15 (m, 2H), 6.72-6.67 (m, 2H), 3.47 (s, 2H), 3.43-3.34 (m, 1H), 3.01-2.92 (m, 8H), 2.15-2.03 (m, 2H), 1.99-1.76 (m, 4H). MS: calcd for C₁₈H₂₃N₃O₂, 313.2; m/z found, 134.1 [4-NMe₂C₆H₆CH₂]⁺.

### Example 21

### [1-(4-Cyclohexyloxy-benzyl)-piperidin-4-yl]-oxazol-2-yl-methanone

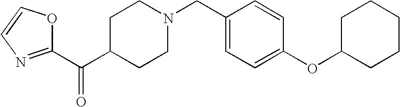

¹H NMR (CDCl₃): 7.81 (d, J=0.5 Hz, 1H), 7.32 (d, J=0.8 Hz, 1H), 7.22-7.18 (m, 2H), 6.87-6.83 (m, 2H), 4.25-4.17 (m, 1H), 3.50-3.35 (m, 4H), 3.00-2.93 (m, 2H), 2.71-1.16 (m, 16H). MS: calcd for C₂₂H₂₈N₂O₃, 368.2; m/z found, 369.1 [M+H]⁺.

### Example 22

### [1-(3-Cyclohexyloxy-benzyl)-piperidin-4-yl]-oxazol-2-yl-methanone

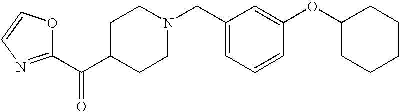

¹H NMR (CDCl₃): 7.82 (d, J=0.5 Hz, 1H), 7.33 (d, J=0.8 Hz, 1H), 7.22-7.17 (m, 1H), 6.91-6.85 (m, 2H), 6.81-6.77 (m, 1H), 4.29-4.21 (m, 1H), 3.50 (s, 2H), 3.45-3.35 (m, 1H), 3.01-2.94 (m, 2H), 2.19-2.07 (m, 2H), 2.03-1.74 (m, 9H), 1.64-1.22 (m, 5H). MS: calcd for C22H28N203, 368.2; m/z found, 369.2 [M+H]⁺.

### Example 23

### [1-(4-Isopropoxy-benzyl)-piperidin-4-yl]-oxazol-2-yl-methanone

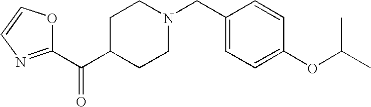

¹H NMR (CDCl₃): 7.81 (d, J=0.8 Hz, 1H), 7.32 (d, J=0.8 Hz, 1H), 7.23-7.18 (m, 2H), 6.86-6.82 (m, 2H), 4.58-4.47 (m, 1H), 3.49-3.34 (m, 3H), 3.00-2.92 (m, 2H), 2.16-2.05 (m, 2H), 2.00-1.77 (m, 4H), 1.33 (d, J=6.1 Hz, 6H). MS: calcd for C₁₉H₂₄N₂O₃, 328.2; m/z found, 329.1 [M+H]⁺.

### Example 24

### [1-(3-Isopropoxy-benzyl)-piperidin-4-yl]-oxazol-2-yl-methanone

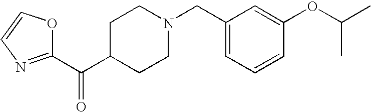

¹H NMR (CDCl₃): 7.82 (d, J=0.5 Hz, 1H), 7.33 (d, J=0.8 Hz, 1H), 7.23-7.18 (m, 1H), 6.90-6.85 (m, 2H), 6.80-6.75 (m, 1H), 4.62-4.51 (m, 1H), 3.50 (s, 2H), 3.45-3.35 (m, 1H), 3.00-2.94 (m, 2H), 2.18-2.08 (m, 2H), 1.99-1.79 (m, 4H). MS: calcd for C₁₉H₂₄N₂O₃, 328.2; m/z found, 329.1 [M+H]⁺.

### Example 25

### [1-(4-Bromo-2-methanesulfonyl-benzyl)-piperidin-4-yl]-oxazol-2-yl-methanone

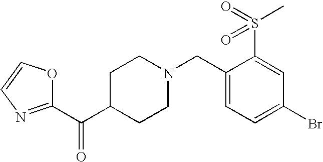

¹H NMR (CDCl₃): 8.25 (d, J=2.0 Hz, 1H), 7.83 (d, J=0.5 Hz, 1H), 7.70-7.67 (m, 1H), 7.34 (d, J=0.5 Hz, 1H), 7.30 (d, J=8.1Hz, 1H), 3.89 (s, 2H), 3.51-3.41 (m, 4H), 3.05-2.98 (m, 2H), 2.28-2.18 (m, 2H), 2.02-1.93 (m, 2H), 1.79-1.65 (m, 2H). MS: calcd for C₁₇H₁₉BrN₂O₄S, 428.0; m/z found, 429.0 [M+H]⁺.

### Example 26

### [1-(4-Bromo-3-nitro-benzyl)-piperidin-4-yl]-oxazol-2-yl-methanone

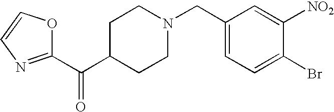

¹H NMR (CDCl₃): 7.84-7.81 (m, 2H), 7.67 (d, J=11.9 Hz, 1H), 7.46-7.42 (m, 1H), 7.34 (d, J=0.5 Hz, 1H), 3.53 (s, 2H), 3.49-3.39 (m, 1H), 2.95-2.88 (m, 2H), 2.24-2.15 (m, 2H), 2.02-1.93 (m, 2H), 1.91-1.78 (m, 2H). MS: calcd for C₁₆H₁₆BrN₃O₄, 393; m/z found, 394 [M+H]⁺.

### Example 27

### [1-(4-Bromo-2-fluoro-benzyl)-piperidin-4-yl]-oxazol-2-yl-methanone

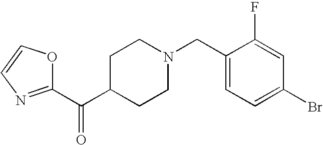

¹H NMR (CDCl₃): 7.82 (d, J=0.8 Hz, 1H), 7.34-7.24 (m, 3H), 7.23-7.20 (m, 1H), 3.55 (d, J=1.0 Hz, 2H), 3.45-3.34 (m, 1H), 2.99-2.91 (m, 2H), 2.24-2.14 (m, 2H), 2.01-1.93 (m, 2H), 1.90-1.77 (m, 2H). MS: calcd for C₁₆H₁₆BrFN₂O₂, 366; m/z found, 367 [M+H]⁺.

### Example 28

### [1-(4-Ethoxy-benzyl)-piperidin-4-yl]-oxazol-2-yl-methanone

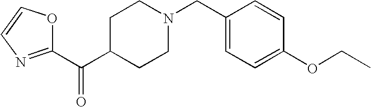

¹H NMR (CDCl₃): 7.81 (d, J=0.5 Hz, 1H), 7.32 (d, J=0.5 Hz, 1H), 7.24-7.20 (m, 2H), 6.87-6.82 (m, 2H), 4.06-3.99 (m, 2H), 3.47 (s, 2H), 3.44-3.35 (m, 1H), 2.99-2.92 (m, 2H), 2.15-2.05 (m, 2H), 1.99-1.91 (m, 2H), 1.89-1.77 (m, 2H), 1.41 (t, J=7.1 Hz, 3H). MS: calcd for C₁₈H₂₂N₂O₃, 314.2; m/z found, 315.1 [M+H]⁺.

### Example 29

### [1-(3-Ethoxy-benzyl)-piperidin-4-yl]-oxazol-2-yl-methanone

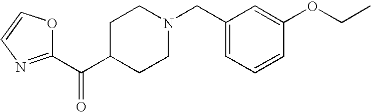

¹H NMR (CDCl₃): 7.82 (d, J=0.8 Hz, 1H), 7.33 (d, J=0.8 Hz, 1H), 7.23-7.19 (m, 1H), 6.92-6.87 (m, 2H), 6.81-6.76 (m, 1H), 4.07-4.01 (m, 2H), 3.50 (s, 2H), 3.45-3.36 (m, 1H), 3.00-2.94 (m, 2H), 2.18-2.08 (m, 2H), 1.99-1.78 (m, 4H), 1.41 (t, 7.0 Hz, 3H). MS: calcd for C₁₈H₂₂N₂O₃, 314.2; m/z found, 315.1 [M+H]⁺.

### Example 30

### [1-(4-Fluoro-benzyl)-piperidin-4-yl]-oxazol-2-yl-methanone

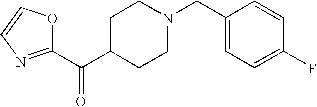

¹H NMR (CDCl₃): 7.82 (d, J=0.5 Hz, 1H), 7.35-7.25 (m, 3H), 7.03-6.96 (m, 2H), 3.49 (s, 2H), 3.45-3.36 (m, 1H), 2.98-2.91 (m, 2H), 2.18-2.07 (m, 2H), 2.00-1.91 (m, 2H), 1.90-1.78 (m, 2H). MS: calcd for C₁₆H₁₇FN₂O₂, 288.1; m/z found, 289.1 [M+H]⁺.

### Example 31

### [1-(3-Fluoro-benzyl)-piperidin-4-yl]-oxazol-2-yl-methanone

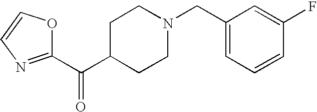

¹H NMR (CDCl₃): 7.82 (d, J=0.8 Hz, 1H), 7.33 (d, J=0.5 Hz, 1H), 7.31-7.23 (m, 1H), 7.11-7.05 (m, 2H), 6.98-6.90 (m, 1H), 3.52 (s, 2H), 3.46-3.37 (m, 1H), 2.99-2.92 (m, 2H), 2.19-2.10 (m, 2H), 2.00-1.80 (m, 4H). MS: calcd for C₁₆H₁₇FN₂O₂, 288.1; m/z found, 289 [M+H]⁺.

### Example 32

### [1-(2-Fluoro-benzyl)-piperidin-4-yl]-oxazol-2-yl-methanone

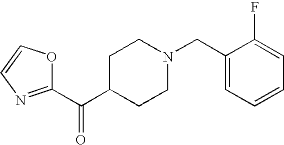

¹H NMR (CDCl₃): 7.81 (d, J=0.5 Hz, 1H), 7.42-7.37 (m, 1H), 7.32 (d, J=0.5 Hz, 1H), 7.25-7.20 (m, 1H), 7.14-7.09 (m, 1H), 7.06-7.00 (m, 1H), 3.62 (d, J=1.0 Hz, 2H), 3.44-3.33 (m, 1H), 3.02-2.96 (m, 2H), 2.25-2.16 (m, 2H), 2.01-1.93 (m, 2H), 1.91-1.79 (m, 2H). MS: calcd for C₁₆H₁₇FN₂O₂, 288.1; m/z found, 289.1 [M+H]⁺.

### Example 33

### [1-(2-Chloro-benzyl)-piperidin-4-yl]-oxazol-2-yl-methanone

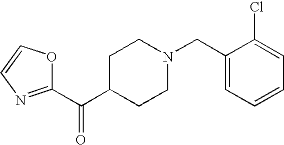

¹H NMR (CDCl₃): 7.82 (d, J=0.8 Hz, 1H), 7.52-7.48 (m, 1H), 7.36-7.33 (m, 2H), 7.25-7.15 (m, 2H), 3.65 (s, 2H), 3.48-3.39 (m, 1H), 3.03-2.97 (m, 2H), 2.30-2.22 (m, 2H), 2.01-1.81 (m, 4H); MS calcd for C₁₆H₁₇ClN₂O₂, 304.1; m/z 305 [M+H]⁺.

### Example 34

### [1-(3-Chloro-benzyl)-piperidin-4-yl]-oxazol-2-yl-methanone

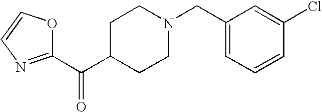

¹H NMR (CDCl₃): 7.82 (d, J=0.5 Hz, 1H), 7.36-7.32 (m, 2H), 7.25-7.18 (m, 3H), 3.50 (s, 2H), 3.46-3.37 (m, 1H), 2.98-2.91 (m, 2H), 2.19-2.09 (m, 2H), 2.00-1.92 (m, 2H), 1.91-1.79 (m, 2H). MS: calcd for C₁₆H₁₇ClN₂O₂, 304.1; m/z found, 305 [M+H]⁺.

### Example 35

### [1-(4-Chloro-benzyl)-piperidin-4-yl]-oxazol-2-yl-methanone

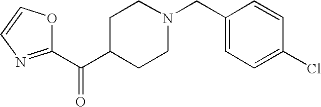

¹H NMR (CDCl₃): 7.82 (d, J=0.8 Hz, 1H), 7.33 (d, J=0.5 Hz, 1H), 7.31-7.24 (m, 4H), 3.49 (s, 2H), 3.45-3.36 (m, 1H), 2.97-2.90 (m, 2H), 2.17-2.08 (m, 2H), 1.99-1.92 (m, 2H), 1.89-1.78 (m, 2H). MS: calcd for C₁₆H₁₇ClN₂O₂, 304.1; m/z found, 305 [M+H]⁺.

### Example 36

### Oxazol-2-yl-[1-(4-trifluoromethoxy-benzyl)-piperidin-4-yl]-methanone

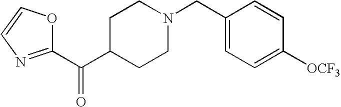

¹H NMR (CDCl₃): 7.82 (d, J=0.5 Hz, 1H), 7.38-7.32 (m, 3H), 7.18-7.14 (m, 2H), 3.52 (s, 2H), 3.46-3.37 (m, 1H), 2.98-2.91 (m, 2H), 2.19-2.10 (m, 2H), 2.00-1.92 (m, 2H), 1.91-1.78 (m, 2H). MS: calcd for C₁₇H₁₇F₃N₂O₃, 354.1; m/z found, 355.1 [M+H]⁺.

### Example 37

### Oxazol-2-yl-[1-(3-trifluoromethoxy-benzyl)-piperidin-4-yl]-methanone

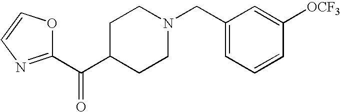

¹H NMR (CDCl₃): 7.82 (d, J=0.5 Hz, 1H), 7.36-7.321 (m, 2H), 7.28-7.20 (m, 2H), 7.13-7.07 (m, 1H), 3.54 (s, 2H), 3.47-3.37 (m, 1H), 2.98-2.91 (m, 2H), 2.20-2.11 (m, 2H), 2.00-1.92 (m, 2H), 1.91-1.80 (m, 2H). MS: calcd for C₁₇H₁₇F₃N₂O₃, 354.1; m/z found, 355.1 [M+H]⁺.

### Example 38

### [1-(3,4-Difluoro-benzyl)-piperidin-4-yl]-oxazol-2-yl-methanone

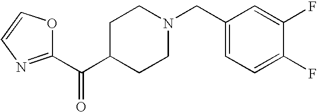

¹H NMR (CDCl₃): 7.83 (s, 1H), 7.33 (s, 1H), 7.23-6.99 (m, 3H), 3.56-3.33 (m, 3H), 2.98-2.88 (m, 2H), 2.20-2.08 (m, 2H), 2.02-1.91 (m, 2H), 1.90-1.77 (m, 2H). MS: calm, for C₁₆H₁₆F₂N₂O₂, 306.1; m/z found, 307.1 [M+H]⁺.

### Example 39

### [1-(1-Methyl-1H-indol-5-ylmethyl)-piperidin-4-yl]-oxazot-2-yl-methanone

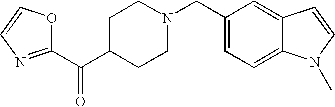

¹H NMR (CDCl₃): 7.80 (s, 1H), 7.54 (s, 1H), 7.32 (s, 1H), 7.30-7.25 (m, 1H), 7.24-7.20 (m, 1H), 7.04 (d, J=3.0 Hz, 1H), 6.46-6.44 (m, 1H), 3.79 (s, 3H), 3.64 (s, 2H), 3.44-3.35 (m, 1H), 3.05-2.98 (m, 2H), 2.19-2.09 (m, 2H), 1.99-1.78 (m, 4H). MS: calcd for C₁₉H₂₁N₃O₂, 323.2; m/z found, 324.1 [M+H]⁺.

### Example 40

### [1-(1-Methyl-1H-indol-6-ylmethyl)-piperidin-4-yl]-oxazol-2-yl-methanone

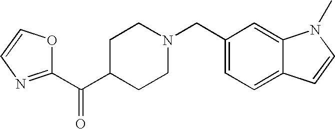

¹H NMR (CDCl₃): 7.81 (d, J=0.8 Hz, 1H), 7.55 (d, J=7.8 Hz, 1H), 7.33-7.30 (m, 2H), 7.09-7.05 (m, 1H), 7.04-7.02 (m, 1H), 6.47-6.45 (m, 1H), 3.80 (s, 3H), 3.46-3.37 (m, 1H), 3.07-3.00 (m, 2H), 2.21-2.11 (m, 2H), 2.01-1.79 (m, 4H). MS: calcd for C₁₉H₂₁N₃O₂, 323.2; m/z found, 324.1 [M+H]⁺.

### Example 41

### (1-[1,8]Naphthyridin-2-ylmethyl-piperidin-4-yl)-oxazol-2-yl-methanone

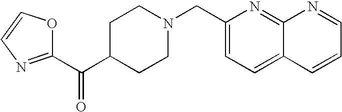

¹H NMR (CDCl₃): 9.11-9.08 (m, 1H), 8.21-8.16 (m, 2H), 7.87-7.81 (m, 2H), 7.50-7.46 (m, 1H), 7.34 (s, 1H), 3.95 (s, 2H), 3.51-3.42 (m, 1H), 3.06-2.99 (m, 2H), 2.42-2.30 (m, 2H), 2.05-1.81 (m, 4H). MS: calcd for C₁₈H₁₈N₄O₂, 322.1; m/z found, 323.1 [M+H]⁺.

### Example 42

### [1-(1H-Indo)-5-ylmethyl)-piperidin-4-yl]-oxazol-2-yl-methanone

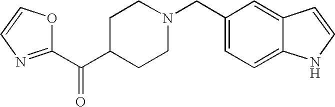

¹H NMR (CDCl₃): 8.17 (s, 1H), 7.81 (d, J=0.5 Hz, 1H), 7.57 (s, 1H), 7.37-7.31 (m, 2H), 7.22-7.17 (m, 2H), 6.54-6.51 (m, 1H), 3.64 (s, 2H), 3.45-3.35 (m, 1H), 3.06-2.99 (m, 2H), 2.20-2.09 (m, 2H), 1.99-1.78 (m, 4H); MS calcd for C₁₈H₁₉N₃O₂, 309.1; m/z found, 310.1 [M+H]⁺.

### Example 43

### [1-(3,4-Dimethoxy-benzyl)-piperidin-4-yl]-oxazol-2-yl-methanone

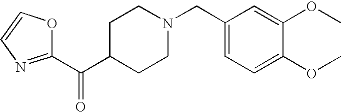

¹H NMR (CDCl₃): 7.82 (d, J=0.8 Hz, 1H), 7.33 (d, J=0.5 Hz, 1H), 6.91 (d, J=1.5 Hz, 1H), 6.85-6.79 (m, 2H), 3.90 (s, 3H), 3.87 (s, 3H), 3.49-3.37 (m, 3H), 3.00-2.93 (m, 2H), 2.16-2.07 (m, 2H), 2.00-1.91 (m, 2H), 1.91-1.78 (m, 2H). MS: calcd for C₁₈H₂₂N₂O₄, 330.2; m/z found, 331.1 [M+H]⁺.

### Example 44

### [1-(3,4-Dichloro-benzyl)-piperidin-4-yl]-oxazol-2-yl-methanone

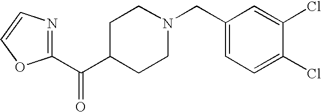

¹H NMR (CDCl₃): 7.82 (d, J=0.8 Hz, 1H), 7.44 (d, J=1.8 Hz, 1H), 7.38 (d, J=8.3 Hz, 1H), 7.33 (d, J=0.8 Hz, 1H), 7.19-7.16 (m, 1H), 3.47-3.38 (m, 3H), 2.95-2.90 (m, 2H), 2.18-2.12 (m, 2H), 1.98-1.94 (m, 2H), 1.90-1.80 (m, 2H). MS: calcd for C₁₆H₁₆Cl₂N₂O₂, 338.1; m/z found, 339.0 [M+H]⁺.

### Example 45

### [1-(1H-Indol-6-ylmethyl)-piperidin-4-yl]-oxazol-2-yl-methanone

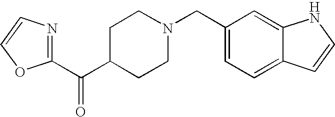

¹H NMR (CDCl₃): 8.13 (bs, 1H), 7.81 (d, J=0.5 Hz, 1H), 7.57 (d, J=8.1 Hz, 1H), 7.39 (s, 1H), 7.32 (d, J=0.8 Hz, 1H), 7.20-7.19 (m, 1H), 7.10-7.07 (m, 1H), 6.54-6.53 (m, 1H), 3.65 (s, 2H), 3.45-3.37 (m, 1H), 3.04-3.00 (m, 2H), 2.19-2.12 (m, 2H), 1.97-1.80 (m, 4H). MS: calcd for C₁₈H₁₉N₃O₂, 309.1; m/z found, 310.1 [M+H]⁺.

### Example 46

### [1-(6-Methoxy-naphthalen-2-ylmethyl)-piperidin-4-yl]-oxazol-2-yl-methanone

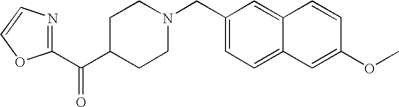

¹H NMR (CDCl₃): 7.82 (d, J=0.8 Hz, 1H), 7.72-7.67 (m, 3H), 7.47-7.45 (m, 1H), 7.32 (d, J=0.5 Hz, 1H), 7.15-7.12 (m, 2H), 3.92 (s, 3H), 3.66 (s, 2H), 3.46-3.38 (m, 1H), 3.03-2.99 (m, 2H), 2.21-2.14 (m, 2H), 1.98-1.82 (m, 4H). MS: calcd for C₂₁H₂₂N₂O₃, 350.2; m/z found, 351.1 [M+H]⁺.

### Example 47

### [1-(1-Hydroxy-naphthalen-2-ylmethyl)-piperidin-4-yl]-oxazol-2-yl-methanone

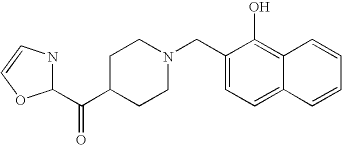

¹H NMR (CDCl₃): 8.25-8.23 (m, 1H), 7.84 (d, J=0.5 Hz, 1H), 7.77-7.73 (m, 1H), 7.48-7.42 (m, 2H), 7.34 (d, J=0.5 Hz, 1H), 7.29 (d, J=8.3 Hz, 1H), 7.08 (d, J=8.3 Hz, 1H), 3.89 (s, 2H), 3.57-3.50 (m, 1H), 3.18-3.14 (m, 2H), 2.37-2.31 (m, 2H), 2.01-1.91 (m, 4H). MS: calcd for C₂₀H₂ₒN₂O₃, 336.1; m/z found, 337.1 [M+H]⁺.

### Example 48

### (1-Naphthalen-1-ylmethyl-piperidin-4-yl)-oxazol-2-yl-methanone

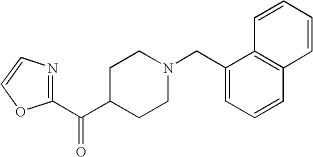

¹H NMR (CDCl₃): 8.33-8.30 (m, 1H), 7.86-7.77 (m, 3H), 7.54-7.38 (m, 4H), 7.33 (d, J=0.8 Hz, 1H), 3.93 (s, 2H), 3.49-3.41 (m, 1H), 3.06-3.02 (m, 2H), 2.25-2.19 (m, 2H), 1.96-1.79 (m, 4H). MS: calcd for C₂₀H₂₀N₂O₂, 320.2; m/z found, 321.1 [M+H]⁺.

### Example 49

### [1-(1-Methyl-1H-benzoimidazol-2-ylmethyl)-piperidin-4-yl]-oxazol-2-yl-methanone

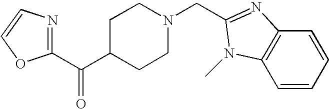

¹H NMR (CDCl₃): 7.82 (d, J=0.8 Hz, 1H), 7.75-7.72 (m, 1H), 7.37-7.23 (m, 4H), 3.89 (s, 3H), 3.85 (s, 2H), 3.50-3.43 (m, 1H), 2.99-2.94 (m, 2H), 2.33-2.26 (m, 2H), 1.98-1.95 (m, 2H), 1.84-1.74 (m, 2H). MS: calcd for C₁₈H₂₀N₄O₂, 324.2; m/z found, 325.1 [M+H]⁺.

### Example 50

### [1-(1-Methyl-1H-indol-2-ylmethyl)-piperidin-4-yl]-oxazol-2-yl-methanone

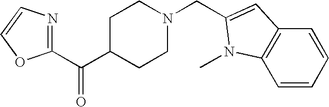

¹H NMR (CDCl₃): 7.82 (d, J=0.5 Hz, 1H), 7.57-7.54 (m, 1H), 7.33-7.30 (m, 2H), 7.21-7.17 (m, 1H), 7.10-7.06 (m, 1H), 6.36 (s, 1H), 3.81 (s, 3H), 3.65 (s, 2H), 3.47-3.40 (m, 1H), 3.02-2.97 (m, 2H), 2.18-2.11 (m, 2H), 1.96-1.92 (m, 2H), 1.83-1.73 (m, 2H). MS: calcd for C₇₃H₂₁N₃O₂, 323.2; m/z found, 324.1 [M+H]⁺.

### Example 51

### [1-(4-Bromo-benzyl)-piperidin-4-yl]-oxazol-2-yl-methanone

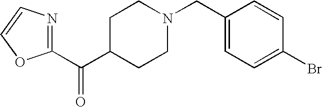

¹H NMR (CDCl₃): 7.82 (d, J=0.8 Hz, 1H), 7.46-7.42 (m, 2H), 7.33 (d, J=0.5 Hz, 1H), 7.23-7.19 (m, 2H), 3.48 (s, 2H), 3.45-3.37 (m, 1H), 2.96-2.91 (m, 2H), 2.16-2.10 (m, 2H), 1.97-1.79 (m, 4H). MS: calcd for C₁₆H₁₇BrN₂O₂, 348; m/z found, 349.0 [M+H]⁺.

### Example 52

### [1-(3-Bromo-benzyl)-piperidin-4-yl]-oxazol-2-yl-methanone

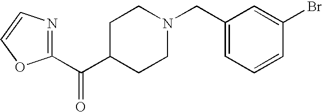

¹H NMR (CDCl₃): 7.82 (d, J=1.0 Hz, 1H), 7.50-7.49 (m, 1H), 7.40-7.37 (m, 1H), 7.33 (d, J=0.3 Hz, 1H), 7.27-7.25 (m, 1H), 7.20-7.16 (m, 1H), 3.50 (s, 2H), 3.46-3.38 (m, 1H), 2.97-2.92 (m, 2H), 2.18-2.11 (m, 2H), 1.98-1.80 (m, 4H). MS: calcd for C₁₆H₁₇BrN₂O₂, 348; m/z found, 349.0 [M+H]⁺.

### Example 53

### [1-(2-Bromo-benzyl)-piperidin-4-yl]-oxazol-2-yl-methanone

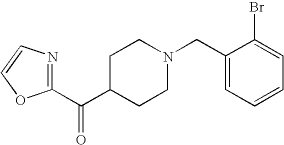

¹H NMR (CDCl₃): 7.83 (d, J=0.5 Hz, 1H), 7.55-7.49 (m, 2H), 7.34 (d, J=0.5 Hz, 1H), 7.31-7.27 (m, 2H), 7.13-7.08 (m, 1H), 3.62 (s, 2H), 3.48-3.40 (m, 1H), 3.02-2.98 (m, 2H), 2.30-2.24 (m, 2H), 1.99-1.82 (m, 4H). MS: calcd for C₁₆H₁₇BrN₂O₂, 348; m/z found, 349.0 [M+H]⁺.

### Example 54

### (1-Benzo[b]thoiphen-2-ylmethyl-piperidin-4-yl)-oxazol-2-yl-methanone

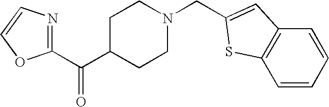

¹H NMR (CDCl₃): 7.82-7.78 (m, 2H), 7.70-7.68 (m, 1H), 7.33-7.27 (m, 3H), 7.15 (d, J=0.8 Hz, 1H), 3.82 (d, J=0.8 Hz, 2H), 3.46-3.39 (m, 1H), 3.09-3.05 (m, 2H), 2.27-2.20 (m, 2H), 2.00-1.84 (m,4H). MS: calcd for C₁₈H₁₈N₂O₂S, 326.1; m/z found, 327.1 [M+H]⁺.

### Example 55

### (1-Benzofuran-2-ylmethyl-piperidin-4-yl)-oxazol-2-yl-methanone

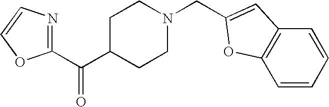

¹H NMR (CDCl₃): 7.81 (d, J=0.5 Hz, 1H), 7.54-7.47 (m, 2H), 7.32 (d, J=0.8 Hz, 1H), 7.28-7.19 (m, 2H), 6.60 (d, J=0.5 Hz, 1H), 3.73 (s, 2H), 3.43-3.36 (m, 1H), 3.09-3.04 (m, 2H), 2.30-2.23 (m, 2H), 2.02-1.86 (m, 4H). MS: calcd for C₁₈H₁₈N₂O₃, 310.1; m/z found, 311.1 [M+H]⁺.

### Example 56

### Oxazol-2-yl-[1-(3-phenoxy-benzyl)-piperidin-4-yl]-methanone

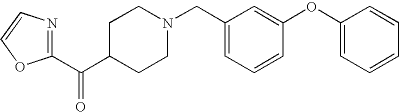

¹H NMR (CDCl₃): 7.82 (d, J=0.5 Hz, 1H), 7.35-7.25 (m, 4H), 7.12-7.07 (m, 2H), 7.02-6.99 (m, 3H), 6.90-6.87 (m, 1H), 3.51 (s, 2H), 3.44-3.36 (m, 1H), 2.98-2.94 (m, 2H), 2.17-2.10 (m, 2H), 1.97-1.78 (m, 4H); MS calcd for C₂₂H₂₂N₂O₃, 362.2; m/z found, 363.1 [M+H]⁺.

### Example 57

### Oxazol-2-yl-[1-(4-phenoxy-benzyl)-piperidin-4-yl]-methanone

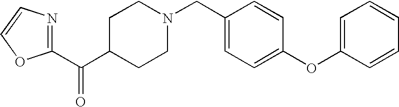

¹H NMR (CDCl₃): 7.82 (s, 1H), 7.35-7.27 (m, 5H), 7.11-7.08 (m, 1H), 7.02-6.95 (m, 4H), 3.51 (s, 2H), 3.45-3.38 (m, 1H), 3.00-2.96 (m, 2H), 2.17-2.11 (m, 2H), 1.99-1.80 (m, 4H). MS: calcd for C₂₂H₂₂N₂O₃, 362.2; m/z found, 363.1 [M +H]⁺.

### Example 58

### Oxazol-2-yl-(1-pyridin-4-ylmethyl-piperidin-4-yl)-methanone

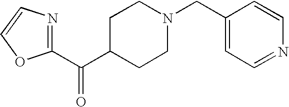

¹H NMR (CDCl₃): 8.55-8.53 (m, 2H), 7.84 (d, J=0.5 Hz, 1H), 7.34 (s, 1H), 7.29-7.28 (m, 2H), 3.53 (s, 2H), 3.47-3.40 (m, 1H), 2.96-2.91 (m, 2H), 2.22-2.15 (m, 2H), 2.05-1.82 (m, 4H). MS: calcd for C₁₅H₁₇N₃O₂, 271.1; m/z found, 272.1 [M+H]⁺.

### Example 59

### Oxazol-2-yl-(1-pyridin-3-ylmethyl-piperidin-4-yl)-methanone

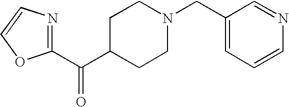

¹H NMR (CDCl₃): 8.54-8.50 (m, 2H), 7.83 (d, J=0.5 Hz, 1H), 7.71-7.68 (m, 1H), 7.33 (d, J=0.5 Hz, 1H), 7.28-7.25 (m, 1H), 3.54 (s, 2H), 3.46-3.38 (m, 1H), 2.97-2.92 (m, 2H), 2.20-2.14 (m, 2H), 1.98-1.79 (m, 4H). MS: calcd for C₁₅H₁₇N₃O₂, 271.1; m/z found, 272.1 [M+H]⁺.

### Example 60

### Oxazol-2-yl-(1-pyridin-2-ylmethyl-piperidin-4-yl)-methanone

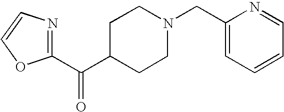

¹H NMR (CDCl₃): 8.57-8.55 (m, 1H), 7.82 (s, 1H), 7.68-7.64 (m, 1H), 7.44 (d, J=7.8 Hz, 1H), 7.33 (s, 1H), 7.18-7.15 (m, 1H), 3.69 (s, 2H), 3.47-3.40 (m 1H), 3.02-2.97 (m, 2H), 2.28-2.22 (m, 2H), 2.00-1.84 (m, 4H). MS: calcd for C₁₅H₁₇N₃O₂, 271.1; m/z found, 272.1 [M+H]⁺.

### Example 61

### [1-(4-Methoxy-benzyl)-piperidin-4-yl]-oxazol-2-yl-methanone

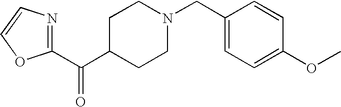

¹H NMR (CDCl₃): 7.13 (d, J=0.5 Hz, 1H), 7.32 (d, J=0.5 Hz, 1H), 7.25-7.22 (m, 2H), 6.88-6.84 (m, 2H), 3.80 (s, 3H), 3.47 (s, 2H), 3.43-3.36 (m, 1H), 2.98-2.93 (m, 2H), 2.14-2.08 (m, 2H), 1.97-1.78 (m, 4H). MS: calcd for C₁₇H₂₀N₂O₃, 300.1; m/z found, 301.1 [M+H]⁺.

### Example 62

### [1-(3-Methoxy-benzyl)-piperidin-4-yl]-oxazol-2-yl-methanone

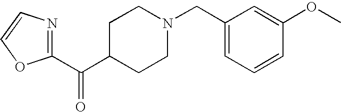

¹H NMR (CDCl₃): 7.82 (d, J=0.5 Hz, 1H), 7.33 (d, J=0.8 Hz, 1H), 7.25-7.21 (m, 1H), 6.92-6.90 (m, 2H), 6.81-6.78 (m, 1H), 3.81 (s, 3H), 3.52 (s, 2H), 3.45-3.37 (m, 1H), 3.00-2.95 (m, 2H), 2.17-2.11 (m, 2H), 1.97-1.80 (m, 4H). MS: calcd for C₁₇H₂₀N₂O₃, 300.1; m/z found, 301.1 [M+H]⁺.

### Example 63

### [1-(2-Methoxy-benzyl)-piperidin-4-yl]-oxazol-2-yl-methanone

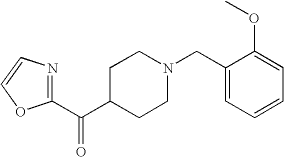

¹H NMR (CDCl₃): 7.81 (d, J=0.5 Hz, 1H), 7.38-7.36 (m, 1H), 7.32 (d, J=0.5 Hz, 1H), 7.26-7.21 (m, 1H), 6.96-6.93 (m, 1H), 6.88-6.86 (m, 1H), 3.83 (s, 3H), 3.60 (s, 2H), 3.43-3.36 (m, 1H), 3.04-3.00 (m, 2H), 2.24-2.17 (m, 2H), 1.98-1.82 (m, 4H). MS: calcd for C₁₇H₂₀N₂O₃, 300.1; m/z found, 301.1 [M+H]⁺.

### Example 64

### [1-(4-Methyl-benzyl)-piperidin-4-yl]-oxazol-2-yl-methanone

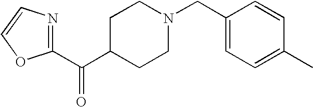

The title compound was prepared in analogy with Example 15, using 4-methylbenzaldehyde. ¹H NMR (CDCl₃): 7.81 (d, J=0.5 Hz, 1H), 7.32 (d, J=0.5 Hz, 1H), 7.21 (d, J=7.8 Hz, 2H), 7.13 (d, J=8.1 Hz, 2H), 3.50 (s, 2H), 3.44-3.36 (m, 1H), 2.99-2.94 (m, 2H), 2.34 (s, 3H), 2.15-2.08 (m, 2H), 1.96-1.79 (m, 4H). MS: calcd for C₁₇H₂₀N₂O₂, 284.2; m/z found, 285.1 [M+H]⁺.

### Example 65

### [1-(3-Methyl-benzyl)-piperidin-4-yl]-oxazol-2-yl-methanone

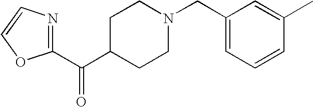

¹H NMR (CDCl₃): 7.82 (d, J=0.8 Hz, 1H), 7.33 (d, J=0.5 Hz, 1H), 7.23-7.06 (m, 4H), 3.50 (s, 2H), 3.44-3.37 (m, 1H), 3.00-2.95 (m, 2H), 2.35 (s, 3H), 2.16-2.10 (m, 2H), 1.97-1.80 (m, 4H). MS: calcd for C₁₇H₂₀N₂O₂, 284.2; m/z found, 285.1 [M+H]⁺.

### Example 66

### [1-(2-Methyl-benzyl)-piperidin-4-yl]-oxazol-2-yl-methanone

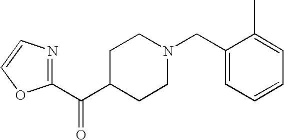

¹H NMR (CDCl₃): 7.82 (d, J=0.8 Hz, 1H), 7.33 (d, J=0.5 Hz, 1H), 7.27-7.26 (m, 1H), 7.17-7.12 (m, 3H), 3.47-3.39 (m, 3H), 2.98-2.93 (m, 2H), 2.36 (s, 3H), 2.18-2.11 (m, 2H), 1.96-1.91 (m, 2H), 1.86-1.76 (m, 2H). MS: calcd for C₁₇H₂₀N₂O₂, 284.2; m/z found, 285.1 [M+H]⁺.

### Example 67

### [1-(6-Chloro-[1,3]dioxolo[4,5-g]quinolin-7-ylmethyl)-piperidin-4-yl]-oxazol-2-yl-methanone

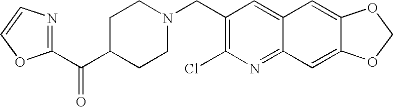

¹H NMR (CDCl₃): 8.08 (s, 1H), 7.84 (d, J=0.8 Hz, 1H), 7.35 (d, J=0.5 Hz, 1H), 7.30 (s, 1H), 7.07. (s, 1H), 6.12 (s, 2H), 3.71 (s, 2H), 3.52-3.45 (m, 1H), 3.06-3.01 (m, 2H), 2.37-2.31 (m, 2H), 2.05-1.87 (m, 4H). MS: calcd for C₂₀H₁₈ClN₃O₄, 399.1; m/z found, 400.1 [M+H]⁺.

### Example 68

### [1-(2-Chloro-7-methoxy-quinolin-3-ylmethyl)-piperidin-4-yl]-oxazol-2-yl-methanone

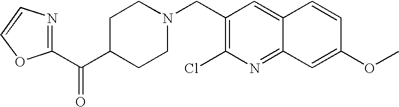

¹H NMR (CDCl₃): 8.18 (s, 1H), 7.84 (d, J=0.8 Hz, 1H), 7.71 (d, J=8.8 Hz, 1H), 7.35-7.34 (m, 2H), 7.22-7.19 (m, 1H), 3.93 (s, 3H), 3.74 (s, 2H), 3.53-3.45 (m, 1H), 3.08-3.03 (m, 2H), 2.38-2.32 (m, 2H), 2.05-1.87 (m, 4H). MS: calcd for C₂₀H₂₀ClN₃O₃, 385.1; m/z found, 386.1 [M+H]⁺.

### Example 69

### [1-(2-Chloro-7-methyl-quinolin-3-ylmethyl)-piperidin-4-yl]-oxazol-2-yl-methanone

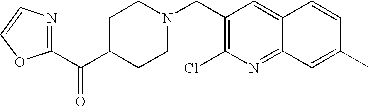

¹H NMR (CDCl₃): 8.22 (s, 1H), 7.84 (d, J=0.8 Hz, 1H), 7.78 (d, J=0.8 Hz, 1H), 7.73 (m, J=8.4 Hz, 1H), 7.40-7.38 (m, 1H), 7.35 (d, J=0.8 Hz, 1H), 3.75 (s, 2H), 3.53-3.45 (m, 1H), 3.08-3.03 (m, 2H), 2.56 (s, 3H), 2.39-2.32 (m, 2H), 2.05-1.88 (m, 4H). MS: calcd for C₂₀H₂₀ClN₃O₂, 369.1; m/z found, 370.1 [M+H]⁺.

### Example 70

### [1-(2,7-Dichloro-quinolin-3-ylmethyl)-piperidin-4-yl]-oxazol-2-yl-methanone

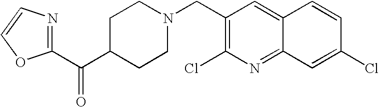

¹H NMR (CDCl₃): 8.27 (s, 1H), 8.00 (d, J=2.0 Hz, 1H), 7.85 (d, J=0.8 Hz, 1H), 7.78 (d, J=8.6 Hz, 1H), 7.53-7.50 (m, 1H), 7.35 (d, J=0.8 Hz, 1H), 3.75 (s, 2H), 3.55-3.47 (m, 1H), 3.06-3.02 (m, 2H), 2.41-2.34 (m, 2H), 2.05-1.89 (m, 4H). MS: calcd for C₁₉H₁₇Cl₂N₃O₂, 389.1; m/z found, 390.1 [M+H]⁺.

### Example 71

### [1-(2,2-Difluoro-benzo[1,3]dioxol-5-ylmethyl)-piperidin-4-yl]-oxazol-2-yl-methanone

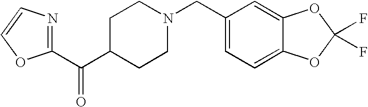

¹H NMR (CDCl₃): 7.82 (d, J=0.5 Hz, 1H), 7.33 (d, J=0.5 Hz, 1H), 7.11 (d, J=1.0 Hz, 1H), 7.02-6.96 (m, 2H), 3.49 (s, 2H), 3.46-3.38 (m, 1H), 2.95-2.90 (m, 2H), 2.17-2.11 (m, 2H), 1.98-1.79 (m, 4H). MS: calcd for C₁₇H₁₆F₂N₂O₄, 350.1; m/z found, 351.1 [M+H]⁺.

### Example 72

### [1-(2-Dimethylamino-quinolin-3-ylmethyl)-piperidin-4-yl]-oxazol-2-yl-methanone

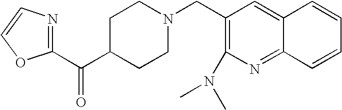

¹H NMR (CDCl₃): 8.13 (s, 1H), 7.83 (d, J=0.8 Hz, 1H), 7.81 (s, 1H), 7.69-7.67 (m, 1H), 7.57-7.53 (m, 1H), 7.34-7.29 (m, 2H), 3.62 (s, 2H), 3.50-3.42 (m, 1H), 3.01-2.98 (m, 8H), 2.26-2.19 (m, 2H), 1.99-1.82 (m, 4H). MS: calcd for C₂₁H₂₄N₄O₂, 364.2; m/z found, 365.2 [M+H]⁺.

### Example 73

### [1-(2-Chloro-8-methyl-quinolin-3-ylmethyl)-piperidin-4-yl]-oxazol-2-yl-methanone

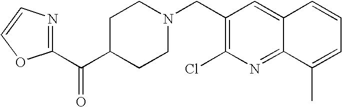

¹H NMR (CDCl₃): 8.22 (s, 1H), 7.84 (d, J=0.8 Hz, 1H), 7.67 (d, J=7.8 Hz, 1H), 7.55-7.53 (m, 1H), 7.46-7.42 (m, 1H), 7.35 (d, J=0.5 Hz, 1H), 3.77 (s, 2H), 3.53-3.45 (m, 1H), 3.08-3.04 (m, 2H), 2.77 (s, 3H), 2.39-2.32 (m, 2H), 2.05-1.88 (m, 4H). MS: calcd for C₂₀H₂₀ClN₃O₂, 369.1; m/z found, 370.1 [M+H]⁺.

### Example 74

### [1-(2-Chloro-6-methyl-quinolin-3-ylmethyl)-piperidin-4-yl]-oxazol-2-yl-methanone

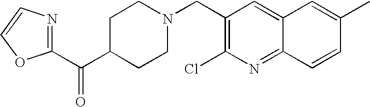

¹H NMR (CDCl₃): 8.18 (s, 1H), 7.90 (d, J=8.6 Hz, 1H), 7.84 (d, J=0.5 Hz, 1H), 7.60 (s, 1H), 7.54-7.52 (m, 1H), 7.35 (d, J=0.5 Hz, 1H), 3.76 (s, 2H), 3.53-3.46 (m, 1H), 3.08-3.03 (m, 2H), 2.54 (s, 3H), 2.39-2.33 (m, 2H), 2.04-1.88 (m, 4H). MS: calcd for C₂₀H₂₀ClN₃O₂, 369.1; m/z found, 370.1 [M+H]⁺.

### Example 75

### [1-(8-Chloro-quinolin-2-ylmethyl)-piperidin-4-yl]-oxazol-2-yl-methanone

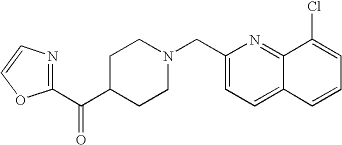

¹H NMR (CDCl₃): 8.15 (d, J=8.3 Hz, 1H), 7.83-7.72 (m, 4H), 7.44-7.41 (m, 1H), 7.34 (d, J=0.8 Hz, 1H), 3.97 (s, 2H), 3.51-3.43 (m, 1H), 3.06-3.01 (m, 2H), 2.39-2.33 (m, 2H), 2.02-1.86 (m, 4H). MS: calcd for C₁₉H₁₈ClN₃O₂, 355.1; m/z found, 356.1 [M+H]⁺.

### Example 76

### [1-(7-Chloro-quinolin-2-ylmethyl)-piperidin-4-yl]-oxazol-2-yl-methanone

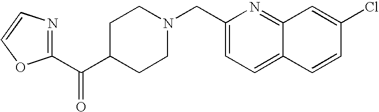

¹H NMR (CDCl₃): 8.11 (d, J=8.6 Hz, 1H), 8.07 (d, J=2.2 Hz, 1H), 7.83 (d, J=0.8 Hz, 1H), 7.74 (d, J=8.3 Hz, 1H), 7.67 (d, J=8.3 Hz, 1H), 7.48-7.46 (m, 1H), 7.34 (d, J=0.5 Hz, 1H), 3.85 (s, 2H), 3.50-3.42 (m, 1H), 3.02-2.98 (m, 2H), 2.35-2.28 (m, 2H), 2.01-1.85 (m, 4H). MS: calcd for C₁₉H₁₈ClN₃O₂, 355.1; m/z found, 356.1 [M+H]⁺.

### Example 77

### [1-(6-Chloro-quinolin-2-ylmethyl)-piperidin-4-yl]-oxazol-2-yl-methanone

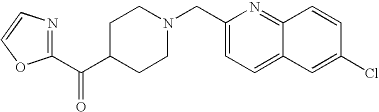

¹H NMR (CDCl₃): 8.05 (d, J=8.3 Hz, 1H), 8.00 (d, J=9.1 Hz, 1H), 7.83 (d, J=0.5 Hz, 1H), 7.79 (d, J=2.5 Hz, 1H), 7.70 (d, J=8.6 Hz, 1H), 7.64-7.61 (m, 1H), 7.33 (d, J=0.8 Hz, 1H), 3.84 (s, 2I-1), 3.50-3.42 (m, 1H), 3.02-2.98 (m, 2H), 2.34-2.28 (m 2H), 2.01-1.85 (m, 4H). MS: calcd for C₁₉H₁₈ClN₃O₂, 355.1; m/z found, 356.1 [M+H]⁺.

### Example 78

### Oxazol-2-yl-(1-quinoxalin-2-ylmethyl-piperidin-4-yl)-methanone

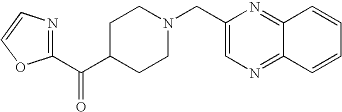

¹H NMR (CDCl₃): 9.05 (s, 1H), 8.12-8.06 (m, 2H), 7.82 (s, 1H), 7.77-7.74 (m, 2H), 7.33 (s, 1H), 3.92 (s, 2H), 3.50-3.42 (m, 1H), 3.02 (d, J=11.9 Hz, 2H), 2.38-2.32 (m, 2H), 2.01-1.86 (m, 4H). MS: calcd for C₁₈F1₁₈N₄O₂, 322.1; m/z found, 323.1 [M+H]⁺.

### Example 79

### [1-(8-Hydroxy-quinolin-2-ylmethyl)-piperidin-4-yl]-oxazol-2-yl-methanone

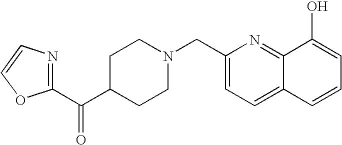

¹H NMR (CDCl₃): 8.12 (d, J=8.6 Hz, 1H), 7.82 (d, J=0.8 Hz, 1H), 7.65 (d, J=8.4 Hz, 1H), 7.41 (d, J=7.3 Hz, 1H), 7.33 (d, J=0.5 Hz, 1H), 7.32-7.30 (m, 1H), 7.17-7.15 (m, 1H), 3.86 (s, 2H), 3.49-3.41 (m, 1H), 3.03-2.99 (m, 2H), 2.34-2.27 (m, 2H), 2.01-1.85 (m, 4H). MS: calcd for C₁₉H₁₉N₃O₃, 337.1; m/z found, 338.1 [M+H]⁺.

### Example 80

### [1-(3-Chloro-quinolin-2-ylmethyl)-piperidin-4-yl]-oxazol-2-yl-methanone

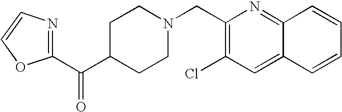

¹H NMR (CDCl₃): 8.29 (s, 1H), 8.01 (d, J=8.6 Hz, 1H), 7.86-7.83 (m, 2H), 7.73-7.69 (m, 1H), 7.58-7.54 (m, 1H), 7.35 (d, J=0.8 Hz, 1H), 3.78 (s, 2H), 3.54-3.47 (m, 1H), 3.09-3.04 (m, 2H), 2.41-2.34 (m, 2H), 2.05-1.89 (m, 4H). MS: calcd for C₁₉H₁₈ClN₃O₂, 355.1; m/z found, 356.1 [M+H]⁺.

### Example 81

### [1-(6-Methoxy-pyridin-3-ylmethyl)-piperidin-4-yl]-oxazol-2-yl-methanone

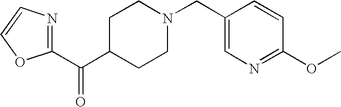

¹H NMR (CDCl₃): 8.03 (s, 1H), 7.82 (s, 1H), 7.60-7.57 (m, 1H), 7.33 (s, 1H), 6.73-6.71 (d, J=8.3 Hz, 1H), 3.93 (s, 3H), 3.46-3.36 (m, 3H), 2.95 (bd, J=11.6 Hz, 2H), 2.16-2.10 (m, 2H), 1.97-1.94 (m, 2H), 1.87-1.77 (m, 2H), 1.30-1.25 (m, 2H). MS: calcd for C₁₈H₁₉N₃O₃, 301.1; m/z found, 302.1 [M+H]⁺.

### Example 82

### [1-(6-Chloro-pyridin-3-ylmethyl)-piperidin-4-yl]-oxazol-2-yl-methanone

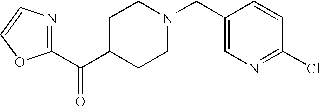

¹H NMR (CDCl₃): 8.30 (d, J=2.0 Hz, 1H), 7.83 (d, J=0.5 Hz, 1H), 7.69-7.67 (m, 1H), 7.33-7.29 (m, 2H), 3.51 (s, 2H), 3.46-3.39 (m, 1H), 2.94-2.89 (m, 2H), 2.20-2.14 (m, 2H), 1.98-1.96 (m, 2H), 1.88-1.78 (m, 2H). MS calcd for C₁₅H₁₆ClN₃O₂, 305.1; m/z found, 306.1 [M+H]⁺.

### Example 83

### [1-(6-Methyl-pyridin-2-ylmethyl)-piperidin-4-yl]-oxazol-2-yl-methanone

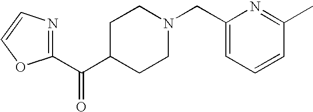

¹H NMR (CDCl₃): 7.82 (s, 1H), 7.57-7.53 (m, 1H), 7.33 (s, 1H), 7.28-7.26 (m, 1H), 7.02 (d, J=7.8 Hz, 1H), 3.66 (s, 2H), 3.47-3.39 (m, 1H), 3.02-2.98 (m, 2H), 2.55 (s, 3H), 2.28-2.21 (m, 2H), 1.99-1.83 (m, 4H). MS calcd for C₁₅H₁₉N₃O₂, 285.1; m/z found, 286.1 [M+H]⁺.

### Example 84

### Oxazol-2-yl-(1-quinolin-2-ylmethyl-piperidin-4-yl)-methanone

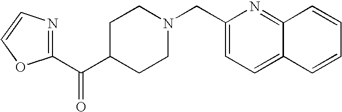

¹H NMR (CDCl₃): 8.14 (d, J=8.6 Hz, 1H), 8.07 (d, J=8.6 Hz, 1H), 7.82-7.80 (m, 2H), 7.72-7.66 (m, 2H), 7.54-7.49 (m, 1H), 7.33 (s, 1H), 3.87 (s, 2H), 3.50-3.42 (m, 1H), 3.00 (d, J=12.1 Hz, 2H), 2.35-2.28 (m, 2H), 2.00-1.85 (m, 4H). MS: calcd for C₁₉H₁₉N₃O₂, 321.1; m/z found, 322.1 [M+H]⁺.

### Example 85

### Oxazol-2-yl-(1-quinolin-3-ylmethyl-piperidin-4-yl)-methanone

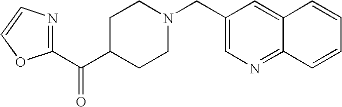

¹H NMR (CDCl₃): 8.89 (d, J=2.3 Hz, 1H), 8.12-8.09 (m, 2H), 7.82-7.80 (m, 2H), 7.71-7.67 (m, 1H), 7.57-7.53 (m, 1H), 7.33 (d, J=0.5 Hz, 1H), 3.72 (s, 2H), 3.48-3.40 (m, 1H), 3.03-2.99 (m, 2H), 2.26-2.20 (m, 2H), 2.00-1.83 (m, 4H). MS: calcd for C₁₉H₁₉N₃O₂, 321.1; m/z found, 322.1 [M+H]⁺.

### Example 86

### [1-(4-Isopropyl-benzyl)-piperidin-4-yl]-oxazol-2-yl-methanone

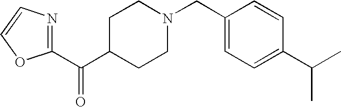

¹H NMR (CDCl₃): 7.81 (d, J=0.5 Hz, 1H), 7.32 (d, J=0.5 Hz, 1H), 7.25-7.23 (m, 2H), 7.19-7.17 (m, 2H), 3.51 (s, 2H), 3.44-3.36 (m, 1H), 3.00-2.84 (m, 3H), 2.16-2.09 (m, 2H), 1.97-1.79 (m, 4H), 1.25 (d, J=7.1 Hz, 6H). MS C₁₉H₂₄N₂O₂, 312.2; m/z found, 313.2 [M+H]⁺.

### Example 87

### [1-(3,4-Dibromo-benzyl)-piperidin-4-yl]-oxazol-2-yl-methanone

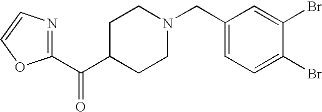

¹H NMR (CDCl₃): 7.83 (d, J=0.5 Hz, 1H), 7.61 (d, J=2.0 Hz, 1H), 7.55 (d, J=8.3 Hz, 1H), 7.33 (d, J=0.5 Hz, 1H), 7.16-7.13 (m, 1H), 3.45-3.38 (m, 3H), 2.94-2.90 (m, 2H), 2.18-2.11 (m, 2H), 1.99-1.79 (m, 4H). MS: calcd for C₁₆H₁₆Br₂N₂O₂, 426; m/z found, 426.9 [M+H]⁺.

### Example 88

### (1-Naphthalen-2-ylmethyl-piperidin-4-yl)-oxazol-2-yl-methanone

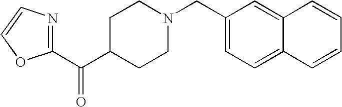

¹H NMR (CDCl₃): 7.84-7.79 (m, 4H), 7.75 (s, 1H), 7.52-7.42 (m, 3H), 7.32 (d, J=0.5 Hz, 1H), 3.70 (s, 2H), 3.47-3.39 (m, 1H), 3.04-3.00 (m, 2H), 2.23-2.16 (m, 2H), 1.98-1.83 (m, 4H). MS: calcd for C₂₀H₂₀N₂O₂, 320.2; m/z found, 321.1 [M+H]⁺.

### Example 89

### (1-Benzyl-piperidin-4-yl)-oxazol-2-yl-methanone

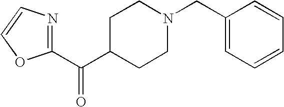

¹H NMR (CDCl₃): 7.81 (s, 1H), 7.33-7.23 (m, 6H), 3.44-3.36 (m, 1H), 2.99-2.94 (m, 2H), 2.17-2.10 (m, 2H), 1.97-1.80 (m, 4H). MS: calcd for C₁₆H₁₈N₂O₂, 270.1; m/z found, 271.1 [M+H]⁺.

### Example 90

### Oxazol-2-yl-[1-(1-phenyl-ethyl)-piperidin-4-yl]-methanone

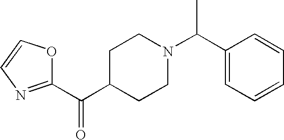

To a solution of Intermediate 3 (65 mg) in acetonitrile (7.5 mL) was added K₂CO₃ (124.4 mg), 1-bromo-1-phenylethane (61.42 mL) and KI (74.7 mg). After 8 h at reflux, the mixture was washed with water (2×7 mL) and extracted with EtOAc (15 mL). The combined organic extracts were dried and concentrated. Purification of the residue by FCC (2 M NH₃ in MeOH/DCM) yielded the title compound (72.8 mg). ¹H NMR (CDCl₃): 7.80 (d, J=0.3 Hz, 1H), 7.33-7.30 (m, 5H), 7.28-7.21 (m, 1H), 3.51-3.44 (m, 1H), 3.39-3.30 (m, 1H), 3.15-3.09 (m, 1H), 2.94-2.88 (m, 1H), 2.20-1.70 (m, 6H), 1.38 (d, J=6.8 Hz, 3H). MS: calcd for C₁₇14₂₀N₂O₂, 284.2; m/z found, 285.1 [M+H]⁺.

### Example 91

### Oxazol-2-yl-{1-[1-(3-trifluoromethyl-phenyl)-ethyl]-piperidin-4-yl}-methanone

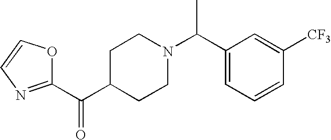

The title compound was prepared using methods analogous to those described in Example 90. ¹H NMR (CDCl₃): 7.81 (d, J=0.5 Hz, 1H), 7.60-7.39 (m, 4H), 7.32 (d, J=0.5 Hz, 1H), 3.58-3.48 (m, 1H), 3.42-3.31 (m, 1H), 3.14-3.04 (m, 1H), 2.90-2.82 (m, 1H), 2.20-1.71 (m, 6H), 1.38 (d, J=6.8 Hz, 3H). MS: calcd for C₁₈H₁₉F₃N₂O₂, 352.1; m/z found, 353.1 [M+H]⁺.

### Biological Testing:

### Assay Method 1

A. Transfection of Cells with Human FAAH

A 10-cm tissue culture dish with a confluent monolayer of SK-N-MC cells was split 2 days (d) prior to transfection. Using sterile technique, the media was removed and the cells were detached from the dish by the addition of trypsin. One fifth of the cells were then placed onto a new 10-cm dish. Cells were grown in a 37° C. incubator with 5% CO₂ in Minimal Essential Media Eagle with 10% Fetal Bovine Serum. After 2 d, cells were approximately 80% confluent. These cells were removed from the dish with trypsin and pelleted in a clinical centrifuge. The pellet was re-suspended in 400 μL complete media and transferred to an electroporation cuvette with a 0.4 cm gap between the electrodes. Supercoiled human FAAH cDNA (1 μg) was added to the cells and mixed. The voltage for the electroporation was set at 0.25 kV, and the capacitance was set at 960 μF. After electroporation, the cells were diluted into complete media (10 mL) and plated onto four 10-cm dishes. Because of the variability in the efficiency of electroporation, four different concentrations of cells were plated. The ratios used were 1:20, 1:10, and 1:5, with the remainder of the cells being added to the fourth dish. The cells were allowed to recover for 24 h before adding the selection media (complete media with 600 μg/mL G418). After 10 d, dishes were analyzed for surviving colonies of cells. Dishes with well-isolated colonies were used. Cells from individual colonies were isolated and tested. The clones that showed the most FAAH activity, as measured by anandamide hydrolysis, were used for further study.

### B. FAAH Assay

T84 frozen cell pellets or transfected SK-N-MC cells (contents of 1×15 cm culture dishes) were homogenized in 50 mL of FAAH assay buffer (125 mM Tris, 1 mM EDTA, 0.2% Glycerol, 0.02% Triton X-100, 0.4 mM Hepes, pH 9). The assay mixture consisted of 50 μL of the cell homogenate, 10 μL of the test compound, and 40 μL of anandamide [1-³H-ethanolamine] (³H-AEA, Perkin-Elmer, 10.3 Cᵢ/mmol), which was added last, for a final tracer concentration of 80 nM. The reaction mixture was incubated at rt for 1 h. During the incubation, 96-well Multiscreen filter plates (catalog number MAFCNOB50; Millipore, Bedford, Mass., USA) were loaded with 25 μL of activated charcoal (Multiscreen column loader, catalog number MACL09625, Millipore) and washed once with 100 μL of MeOH. Also during the incubation, 96-well DYNEX MicroLite plates (catalog number NL510410) were loaded with 100 μL of MicroScint40 (catalog number 6013641, Packard Bioscience, Meriden, Conn., USA). After the 1 h incubation, 60 μL of the reaction mixture were transferred to the charcoal plates, which were then assembled on top of the DYNEX plates using Centrifuge Alignment Frames (catalog number MACF09604, Millipore). The unbound labeled ethanolamine was centrifuged through to the bottom plate (5 min at 2000 rpm), which was preloaded with the scintillant, as described above. The plates were sealed and left at rt for 1 h before counting on a Hewlett Packard TopCount.

### Assay Method 2

A. Transfection of Cells with Rat FAAH

A 10-cm tissue culture dish with a confluent monolayer of SK-N-MC cells was split 2 days (d) prior to transfection. Using sterile technique, the media was removed and the cells were detached from the dish by the addition of trypsin. One fifth of the cells were then placed onto a new 10-cm dish. Cells were grown in a 37° C. incubator with 5% CO₂ in Minimal Essential Media Eagle with 10% Fetal Bovine Serum. After 2 d, cells were approximately 80% confluent. These cells were removed from the dish with trypsin and pelleted in a clinical centrifuge. The pellet was re-suspended in 400 μL complete media and transferred to an electroporation cuvette with a 0.4 cm gap between the electrodes. Supercoiled rat FAAH cDNA (1 μg) was added to the cells and mixed. The voltage for the electroporation was set at 0.25 kV, and the capacitance was set at 960 μF. After electroporation, the cells were diluted into complete media (10 mL) and plated onto four 10-cm dishes. Because of the variability in the efficiency of electroporation, four different concentrations of cells were plated. The ratios used were 1:20, 1:10, and 1:5, with the remainder of the cells being added to the fourth dish. The cells were allowed to recover for 24 h before adding the selection media (complete media with 600 μg/mL G418). After 10 d, dishes were analyzed for surviving colonies of cells. Dishes with well-isolated colonies were used. Cells from individual colonies were isolated and tested. The clones that showed the most FAAH activity, as measured by anandamide hydrolysis, were used for further study.

### B. FAAH Assay

T84 frozen cell pellets or transfected SK-N-MC cells (contents of 1×15 cm culture dishes) were homogenized in 50 mL of FAAH assay buffer (125 mM Tris, 1 mM EDTA, 0.2% Glycerol, 0.02% Triton X-100, 0.4 mM Hepes, pH 9). The assay mixture consisted of 50 μL of the cell homogenate, 10 μL of the test compound, and 40 μL of anandamide [1-³H-ethanolamine] (³H-AEA, Perkin-Elmer, 10.3 Cᵢ/mmol), which was added last, for a final tracer concentration of 80 nM. The reaction mixture was incubated at it for 1 h. During the incubation, 96-well Multiscreen filter plates (catalog number MAFCNOB50; Millipore, Bedford, Mass., USA) were loaded with 25 μL of activated charcoal (Multiscreen column loader, catalog number MACL09625, Millipore) and washed once with 100 μL of MeOH. Also during the incubation, 96-well DYNEX MicroLite plates (catalog number NL510410) were loaded with 100 μL of MicroScint40 (catalog number 6013641, Packard Bioscience, Meriden, Conn., USA). After the 1 h incubation, 60 μL of the reaction mixture were transferred to the charcoal plates, which were then assembled on top of the DYNEX plates using Centrifuge Alignment Frames (catalog number MACF09604, Millipore). The unbound labeled ethanolamine . was centrifuged through to the bottom plate (5 min at 2000 rpm), which was preloaded with the scintillant, as described above. The plates were sealed and left at rt for 1 h before counting on a Hewlett Packard TopCount.

Results for compounds tested in these assays are presented in Table 1. Where activity is shown as greater than (>) a particular value, the value is the solubility limit of the compound in the assay medium or the highest concentration tested in the assay.

TABLE 1

| | Assay 1 | Assay 2 |
|---|---|---|
| Ex. | IC₅₀ (μM) | IC₅₀ (μM) |
| 1 | 0.100 | 1.00 |
| 2 | 10.0 | >10 |
| 3 | 8.0 | 10.0 |
| 4 | 1.20 | 8.00 |
| 5 | 8.00 | 8.00 |
| 6 | 5.00 | 10.0 |
| 7 | 10.0 | >10 |
| 8 | >10 | >10 |
| 9 | 7.00 | >10 |
| 10 | 0.028 | 0.350 |
| 11 | 2.15 | 7.50 |
| 12 | 9.00 | 10.0 |
| 13 | 10.0 | >10 |
| 14 | 1.60 | 9.00 |
| 15 | 0.113 | 0.582 |
| 16 | 0.031 | 0.975 |
| 17 | 0.180 | 10.0 |
| 18 | 0.051 | 0.096 |
| 19 | 0.010 | 0.015 |
| 20 | 0.200 | 10.0 |
| 21 | 0.750 | 10.0 |
| 22 | 0.012 | 0.790 |
| 23 | 5.00 | >10 |
| 24 | 0.118 | 10.0 |
| 25 | 0.013 | 0.023 |
| 26 | 0.003 | 0.009 |
| 27 | 0.011 | 0.075 |
| 28 | 0.772 | 10.0 |
| 29 | 0.170 | 1.40 |
| 30 | 0.148 | 0.873 |
| 31 | 0.303 | 1.40 |
| 32 | 1.50 | >10 |
| 33 | 0.156 | 0.890 |
| 34 | 0.078 | 0.300 |
| 35 | 0.019 | 0.040 |
| 36 | 0.025 | 0.040 |
| 37 | 0.025 | 0.040 |
| 38 | 0.030 | 0.076 |
| 39 | 0.080 | 2.00 |
| 40 | 0.094 | 0.385 |
| 41 | 0.309 | 1.75 |
| 42 | 0.519 | >10 |
| 43 | 3.00 | 6.00 |
| 44 | 0.005 | 0.005 |
| 45 | 0.212 | 1.60 |
| 46 | 0.260 | 0.219 |
| 47 | 0.004 | 0.007 |
| 48 | 0.101 | 0.063 |
| 49 | 0.037 | 0.091 |
| 50 | 0.004 | 0.008 |
| 51 | 0.012 | 0.033 |
| 52 | 0.035 | 0.200 |
| 53 | 0.085 | 0.320 |
| 54 | 0.002 | 0.009 |
| 55 | 0.012 | 0.105 |
| 56 | 0.028 | 0.132 |
| 57 | 0.210 | 0.532 |
| 58 | 10.0 | >10 |
| 59 | 10.0 | >10 |
| 60 | 1.30 | >10 |
| 61 | 0.208 | 1.20 |
| 62 | 0.244 | 1.30 |
| 63 | 10.0 | 10.0 |
| 64 | 0.112 | 0.484 |
| 65 | 0.250 | 0.970 |
| 66 | 0.175 | 0.600 |
| 67 | 0.745 | >10 |
| 68 | 0.294 | 0.511 |
| 69 | 0.585 | 0.470 |
| 70 | 0.500 | 0.290 |
| 71 | 0.027 | 0.024 |
| 72 | 0.046 | 0.038 |
| 73 | 0.002 | 0.004 |
| 74 | 0.017 | 0.087 |
| 75 | 0.004 | 0.004 |
| 76 | 0.003 | 0.008 |
| 77 | 0.007 | 0.022 |
| 78 | 0.030 | 0.090 |
| 79 | 0.012 | 0.040 |
| 80 | 0.016 | 0.070 |
| 81 | 0.315 | 1.70 |
| 82 | 0.125 | 0.650 |
| 83 | 0.640 | 3.00 |
| 84 | 0.013 | 0.023 |
| 85 | 0.018 | 0.043 |
| 86 | 0.110 | 0.170 |
| 87 | 0.002 | 0.002 |
| 88 | 0.004 | 0.009 |
| 89 | 3.00 | 7.00 |
| 90 | 10.0 | >10 |
| 91 | 0.782 | 0.370 |

While the invention has been illustrated by reference to exemplary and preferred embodiments, it will be understood that the invention is intended not to be limited to the foregoing detailed description, but to be defined by the appended claims as properly construed under principles of patent law.

What is claimed is:

1. A chemical entity selected from compounds of Formula (I): 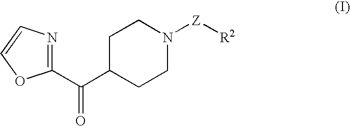
wherein:
Z is —C(O)(CH₂)ₙ—, —SO₂—, or —CH(R^{f})—;
where n is 0, 1, or 2; and
R^{f} is H or C₁₋₄alkyl; and
R² is:
(a) a phenyl group, unsubstituted or substituted with one, two, or three R^{a} moieties;
where each R^{a} moiety is: independently selected from the group consisting of C₁₋₇alkyl, —C₃₋₇cycloalkyl, —C₂₋₇alkenyl, —OH, —OC₁₋₇alkyl, —OC₃₋₇cycloalkyl, phenyl unsubstituted or substituted with R^{b}, phenoxy unsubstituted or substituted with R^{b}, halo, —CF₃, —OCF₃, —SC₁₋₄alkyl, —SO₂C₁₋₄alkyl, —SOC₁₋₄alkyl, —CN, —NO₂, —CO₂C₁₋₄alkyl, —CO₂H, —SO₂NR^{c}R^{d}, —NR^{c}SO₂R^{d}, —C(O)NR^{c}R^{d}, —NR^{c}C(O)R^{d}, and —N(R^{c})R^{d}; or two adjacent R^{a} moieties together form —O(CH₂)₁₋₂O— or —O(CF₂)O—;
where R^{b} is selected from the group consisting of —C₁₋₄alkyl, —OC₁₋₄alkyl, halo, —CN, —OH, —CF₃, —OCF₃, and —NO₂; and
where R^{c} and R^{d} are each independently —H or —C₁₋₇alkyl;
(b) a five- or six-membered monocyclic heteroaryl group, unsubstituted or substituted with one or two R^{a} moieties;
(c) a naphthyl group, unsubstituted or substituted with one or two R^{e} moieties;
where each R^{e} moiety is: independently selected from the group consisting of —C₁₋₄alkyl, —OC₁₋₄alkyl, halo, —CN, —OH, —CF₃, —OCF₃, and —NO₂; or two adjacent R^{e} moieties together form —O(CH₂)₁₋₂O— or —O(CF₂)O—; or
(d) a nine- or ten-membered fused bicyclic heteroaryl group, unsubstituted or substituted with one or two R^{e} moieties;
and pharmaceutically acceptable salts of compounds of Formula (I),
pharmaceutically acceptable prodrugs of compounds of Formula (I), and
pharmaceutically active metabolites of Formula (I).

2. A chemical entity as defined in claim 1, wherein Z is —C(O)—, —SO₂—, or —CH₂—.

3. A chemical entity as defined in claim 1, wherein n is 2.

4. A chemical entity as defined in claim 1, wherein R^{f} is H or CH₃.

5. A chemical entity as defined in claim 1, wherein Z is —CH₂—.

6. A chemical entity as defined in claim 1 or 2, wherein R² is a phenyl, pyrrolyl, furanyl, thiophenyl, pyrazolyl, imidazolyl, oxazolyl, isoxazolyl, thiazolyl, oxadiazolyl, thiadiazolyl, pyridyl, pyridazinyl, pyrimidinyl, or pyrazinyl group, unsubstituted or substituted with one, two, or three of the R^{a} moieties.

7. A chemical entity as defined in claim 1, wherein R² is a phenyl group, unsubstituted or substituted with one, two, or three of the R^{a} moieties.

8. A chemical entity as defined in claim 1, wherein R² is a naphthyl, benzofuranyl, benzothiophenyl, indolyl, benzoimidazolyl, benzoxazolyl, benzothiazolyl, quinolinyl, isoquinolinyl, or naphthyridinyl group, unsubstituted or substituted with one or two of the R^{e} moieties.

9. A chemical entity as defined in claim 1, wherein R² is a naphthyl, benzofuranyl, benzothiophenyl, indolyl, benzoimidazolyl, quinolinyl, or naphthyridinyl group, unsubstituted or substituted with one or two of the R^{e} moieties.

10. A chemical entity as defined in claim 1, wherein each R^{a} moiety is: independently selected from the group consisting of methyl, ethyl, propyl, isopropyl, 2-methylpropyl, cyclopropyl, cyclobutyl, cyclopentyl, cyclohexyl, —OH, methoxy, ethoxy, isopropoxy, isobutoxy, cyclopentyloxy, cyclohexyloxy, phenyl unsubstituted or substituted with R^{b}, phenoxy unsubstituted or substituted with R^{b}, fluoro, chloro, bromo, —CF₃, —OCF₃, methanesulfanyl, methanesulfonyl, —CN, —NO₂, methoxycarbonyl, ethoxycarbonyl, —CO₂H, acetyl, —SO₂NR^{c}R^{d}, —NR^{c}SO₂R^{d}, —C(O)NR^{c}R^{d}, —NR^{c}C(O)R^{d}, and —N(R^{c})R^{d}; or two adjacent R^{a} moieties together form —O(CH₂)₁₋₂O— or —O(CF₂)O—.

11. A chemical entity as defined in claim 10, wherein R^{b} is selected from the group consisting of methyl, ethyl, isopropy, methoxy, ethoxy, fluoro, chloro, bromo, —CN, —OH, —CF₃, —OCF₃, and —NO₂.

12. A chemical entity as defined in claim 10, wherein R^{c} and R^{d} are each independently H, methyl, ethyl, or isopropyl.

13. A chemical entity as defined in claim 1, wherein each R^{e} moiety is:
independently selected from the group consisting of methyl, ethyl, propyl, isopropyl, methoxy, ethoxy, propoxy, isopropoxy, fluoro, chloro, bromo, —CN, —OH, —CF₃, —OCF₃, and —NO₂; or two adjacent R^{e} moieties together form —O(CH₂)₁₋₂O— or —O(CF₂)O—.

14. A chemical entity as defined in claim 1, wherein R² is phenyl, 2-methylphenyl, 4-methylphenyl, 3-methylphenyl, 4-ethylphenyl, 4-isopropylphenyl, 4-isobutylphenyl, 4-t-butylphenyl, 4-cyclohexylphenyl, 2-methoxyphenyl, 3-methoxyphenyl, 4-methoxyphenyl, 3-ethoxyphenyl, 4-ethoxyphenyl, 3-isopropoxyphenyl, 4-isopropoxyphenyl, 3-isobutyoxphenyl, 4-isobutoxyphenyl, 4-t-butoxyphenyl, 3-cyclohexyloxyphenyl, 4-cyclohexyloxyphenyl, 3-biphenyl, 4-biphenyl, 3-phenoxyphenyl, 4-phenoxyphenyl, 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 3-trifluoromethylphenyl, 4-trifluoromethylphenyl, 3-trifluoromethoxyphenyl, 4-trifluoromethoxyphenyl, 4-dimethylaminophenyl, 4-diethylaminophenyl, 2,3-dimethylphenyl, 3,4-dimethoxyphenyl, 2,3-difluorophenyl, 2,4-difluorophenyl, 2,5-difluorophenyl, 3,4-difluorophenyl, 3,4-dichlorophenyl, 3,4-dibromophenyl, 4-bromo-2-fluorophenyl, 3-chloro-4-fluorophenyl, 2,4,6-trifluorophenyl, 2,3,5-trifluorophenyl, 4-bromo-2-methanesulfanylphenyl, 4-bromo-3-nitrophenyl, benzo[1,3]dioxolyl, 2,2-difluoro-benzo[1,3]dioxol-5-yl, 2-furanyl, 3-methyl-isoxazol-5-yl, pyridin-2-yl, pyridin-3-yl, pyridin-4-yl, 6-methyl-pyridin-2-yl, 6-bromo-pyridin-2-yl, 6-methoxy-pyridin-3-yl, 6-chloro-pyridin-3-yl, 5-bromo-pyridin-3-yl, 6-bromo-pyridin-3-yl, 6-phenoxy-pyridin-3-yl, 6-p-tolyloxy-pyridin-3-yl, 6-(3-methoxy-phenyl)-pyridin-3-yl, 6-(3-cyanophenyl)-pyridin-3-yl, napthalen-1-yl, naphthalen-2-yl, 1-hydroxy-naphthalen-2-yl, 6-methoxy-naphthalen-2-yl, 1-methyl-1H-indol-2-yl, 1H-indol-5-yl, 1-methyl-1H-indol-5-yl, 1H-indol-6-yl, 1-methyl-1H-indol-6-yl, benzofuran-2-yl, benzo[b]thiophen-2-yl, 1-methyl-1H-benzoimidazol-2-yl, 2-quinolinyl, 3-quinolinyl, 4-quinolinyl, 3-chloro-quinolin-2-yl, 6-chloro-quinolin-2-yl, 7-chloro-quinolin-2-yl, 8-chloro-quinolin-2-yl, 8-hydroxy-quinolin-2-yl, 2-chloro-quinolin-3-yl, 2-dimethylamino-quinolin-3-yl, 2-chloro-6-methyl-quinolin-3-yl, 2-chloro-8-methyl-quinolin-3-yl, 2-chloro-6-methoxy-quinolin-3-yl, 2-chloro-7-methoxy-quinolin-3-yl, 2-chloro-7-methyl-quinolin-3-yl, 2,7-dichloro-quinolin-3-yl, 6-chloro-[1,3]dioxolo[4,5-g]quinolin-7-yl, [1,8]naphthyridin-2-yl, or quinoxalin-2-yl.

15. A chemical entity as defined in claim 2, wherein R² is phenyl, 2-methylphenyl, 4-methylphenyl, 3-methylphenyl, 4-ethylphenyl, 4-isopropylphenyl, 4-isobutylphenyl, 4-t-butylphenyl, 4-cyclohexylphenyl, 2-methoxyphenyl, 3-methoxyphenyl, 4-methoxyphenyl, 3-ethoxyphenyl, 4-ethoxyphenyl, 3-isopropoxyphenyl, 4-isopropoxyphenyl, 3-isobutyoxphenyl, 4-isobutoxyphenyl, 4-t-butoxyphenyl, 3-cyclohexyloxyphenyl, 4-cyclohexyloxyphenyl, 3-biphenyl, 4-biphenyl, 3-phenoxyphenyl, 4-phenoxyphenyl, 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 3-trifluoromethylphenyl, 4-trifluoromethylphenyl, 3-trifluoromethoxyphenyl, 4-trifluoromethoxyphenyl, 4-dimethylaminophenyl, 4-diethylaminophenyl, 2,3-dimethylphenyl, 3,4-dimethoxyphenyl, 2,3-difluorophenyl, 2,4-difluorophenyl, 2,5-difluorophenyl, 3,4-difluorophenyl, 3,4-dichlorophenyl, 3,4-dibromophenyl, 4-bromo-2-fluorophenyl, 3-chloro-4-fluorophenyl, 2,4,6-trifluorophenyl, 2,3,5-trifluorophenyl, 4-bromo-2-methanesulfanylphenyl, 4-bromo-3-nitrophenyl, benzo[1,3]dioxolyl, 2,2-difluoro-benzo[1,3]dioxol-5-yl, 2-furanyl, 3-methyl-isoxazol-5-yl, pyridin-2-yl, pyridin-3-yl, pyridin-4-yl, 6-methyl-pyridin-2-yl, 6-bromo-pyridin-2-yl, 6-methoxy-pyridin-3-yl, 6-chloro-pyridin-3-yl, 5-bromo-pyridin-3-yl, 6-bromo-pyridin-3-yl, 6-phenoxy-pyridin-3-yl, 6-p-tolyloxy-pyridin-3-yl, 6-(3-methoxy-phenyl)-pyridin-3-yl, 6-(3-cyanophenyl)-pyridin-3-yl, napthalen-1-yl, naphthalen-2-yl, 1-hydroxy-naphthalen-2-yl, 6-methoxy-naphthalen-2-yl, 1-methyl-1H-indol-2-yl, 1H-indol-5-yl, 1-methyl-1H-indol-5-yl, 1-methyl-1H-indol-6-yl, benzofuran-2-yl, benzo[b]thiophen-2-yl, 1-methyl-1H-benzoimidazol-2-yl, 2-quinolinyl, 3-quinolinyl, 4-quinolinyl, 3-chloro-quinolin-2-yl, 7-chloro-quinolin-2-yl, 8-chloro-quinolin-2-yl, 8-hydroxy-quinolin-2-yl, 2-chloro-quinolin-3-yl, 2-dimethylamino-quinolin-3-yl, 2-chloro-6-methyl-quinolin-3-yl, 2-chloro-8-methyl-quinolin-3-yl, 2-chloro-6-methoxy-quinolin-3-yl, 2-chloro-7-methoxy-quinolin-3-yl, 2-chloro-7-methyl-quinolin-3-yl, 2,7-dichloro-quinolin-3-yl, 6-chloro-[1,3]dioxolo[4,5-g]quinolin-7-yl, [1,8]naphthyridin-2-yl, or quinoxalin-2-yl.

16. A chemical entity as defined in claim 5, wherein R² is phenyl, 2-methylphenyl, 4-methylphenyl, 3-methylphenyl, 4-ethylphenyl, 4-isopropylphenyl, 4-isobutylphenyl, 4-t-butylphenyl, 4-cyclohexylphenyl, 2-methoxyphenyl, 3-methoxyphenyl, 4-methoxyphenyl, 3-ethoxyphenyl, 4-ethoxyphenyl, 3-isopropoxyphenyl, 4-isopropoxyphenyl, 3-isobutyoxphenyl, 4-isobutoxyphenyl, 4-t-butoxyphenyl, 3-cyclohexyloxyphenyl, 4-cyclohexyloxyphenyl, 3-biphenyl, 4-biphenyl, 3-phenoxyphenyl, 4-phenoxyphenyl, 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 3-trifluoromethylphenyl, 4-trifluoromethylphenyl, 3-trifluoromethoxyphenyl, 4-trifluoromethoxyphenyl, 4-dimethylaminophenyl, 4-diethylaminophenyl, 2,3-dimethylphenyl, 3,4-dimethoxyphenyl, 2,3-difluorophenyl, 2,4-difluorophenyl, 2,5-difluorophenyl, 3,4-difluorophenyl, 3,4-dichlorophenyl, 3,4-dibromophenyl, 4-bromo-2-fluorophenyl, 3-chloro-4-fluorophenyl, 2,4,6-trifluorophenyl, 2,3,5-trifluorophenyl, 4-bromo-2-methanesulfanylphenyl, 4-bromo-3-nitrophenyl, benzo[1,3]dioxolyl, 2,2-difluoro-benzo[1,3]dioxol-5-yl, 2-furanyl, 3-methyl-isoxazol-5-yl, pyridin-2-yl, pyridin-3-yl, pyridin-4-yl, 6-methyl-pyridin-2-yl, 6-bromo-pyridin-2-yl, 6-methoxy-pyridin-3-yl, 6-chloro-pyridin-3-yl, 5-bromo-pyridin-3-yl, 6-bromo-pyridin-3-yl, 6-phenoxy-pyridin-3-yl, 6-p-tolyloxy-pyridin-3-yl, 6-(3-methoxy-phenyl)-pyridin-3-yl, 6-(3-cyanophenyl)-pyridin-3-yl, napthalen-1-yl, naphthalen-2-yl, 1-hydroxy-naphthalen-2-yl, 6-methoxy-naphthalen-2-yl, 1-methyl-1H-indol-2-yl, 1H-indol-5-yl, 1-methyl-1H-indol-5-yl, 1H-indol-6-yl, 1-methyl-1H-indol-6-yl, benzofuran-2-yl, benzo[b]thiophen-2-yl, 1-methyl-1H-benzoimidazol-2-yl, 2-quinolinyl, 3-quinolinyl, 4-quinolinyl, 3-chloro-quinolin-2-yl, 6-chloro-quinolin-2-yl, 7-chloro-quinolin-2-yl, 8-chloro-quinolin-2-yl, 8-hydroxy-quinolin-2-yl, 2-chloro-quinolin-3-yl, 2-dimethylamino-quinolin-3-yl, 2-chloro-6-methyl-quinolin-3-yl, 2-chloro-8-methyl-quinolin-3-yl, 2-chloro-6-methoxy-quinolin-3-yl, 2-chloro-7-methoxy-quinolin-3-yl, 2-chloro-7-methyl-quinolin-3-yl, 2,7-dichloro-quinolin-3-yl, 6-chloro-[1,3]dioxolo[4,5-g]quinolin-7-yl, [1,8]naphthyridin-2-yl, or quinoxalin-2-yl.

17. A chemical entity as defined in claim 5, wherein R² is benzo[1,3]dioxolyl or 2,2-difluoro-benzo[1,3]dioxol-5-yl.

18. A chemical entity as defined in claim 5, wherein R² is a phenyl group substituted with one or two R^{a} moieties, where each R^{a} moiety is independently selected from halo.

19. A chemical entity as defined in claim 8, wherein Z is —CH₂—.

20. A chemical entity selected from the group consisting of:
[1-(Naphthalene-2-sulfonyl)-piperidin-4-yl]-oxazol-2-yl-methanone;
[1-(4-Methoxy-benzenesulfonyl)-piperidin-4-yl]-oxazol-2-yl-methanone;
[1-(3-Methoxy-benzenesulfonyl)-piperidin-4-yl]-oxazol-2-yl-methanone;
[1-(4-Chloro-benzenesulfonyl)-piperidin-4-yl]-oxazol-2-yl-methanone;
[1-(3-Chloro-benzenesulfonyl)-piperidin-4-yl]-oxazol-2-yl-methanone;
[1-(2-Chloro-benzenesulfonyl)-piperidin-4-yl]-oxazol-2-yl-methanone;
[1-(3-Methoxy-benzoyl)-piperidin-4-yl]-oxazol-2-yl-methanone;
[1-(2-Methoxy-benzoyl)-piperidin-4-yl]-oxazol-2-yl-methanone;
[1-(4-Methoxy-benzoyl)-piperidin-4-yl]-oxazol-2-yl-methanone;
[1-(Naphthalene-2-carbonyl)-piperidin-4-yl]-oxazol-2-yl-methanone;
[1-(4-Chloro-benzoyl)-piperidin-4-yl]-oxazol-2-yl-methanone;
[1-(3-Chloro-benzoyl)-piperidin-4-yl]-oxazol-2-yl-methanone;
[1-(2-Chloro-benzoyl)-piperidin-4-yl]-oxazol-2-yl-methanone:
1-[4-(Oxazole-2-carbonyl)-piperidin-1-yl]-3-phenyl-propan-1-one;
(1-Benzo[1,3]dioxol-5-ylmethyl-piperidin-4-yl)-oxazol-2-yl-methanone;
[1-(3-Isobutoxy-benzyl)-piperidin-4-yl]-oxazol-2-yl-methanone;
[1-(4-Isobutoxy-benzyl)-piperidin-4-yl]-oxazol-2-yl-methanone;
Oxazol-2-yl-[1-(3-trifluoromethyl-benzyl)-piperidin-4-yl]-methanone;
Oxazol-2-yl-[1-(4-trifluoromethyl-benzyl)-piperidin-4-yl]-methanone;
[1-(4-Dimethylamino-benzyl)-piperidin-4-yl]-oxazol-2-yl-methanone;
[1-(4-Cyclohexyloxy-benzyl)-piperidin-4-yl]-oxazol-2-yl-methanone;
[1-(3-Cyclohexyloxy-benzyl)-piperidin-4-yl)-oxazol-2-yl-methanone;
[1-(4-Isopropoxy-benzyl)-piperidin-4-yl]-oxazol-2-yl-methanone;
[1-(3-Isopropoxy-benzyl)-piperidin-4-yl]-oxazol-2-yl-methanone;
[1-(4-Bromo-2-methanesulfonyl-benzyl)-piperidin-4-yl]-oxazol-2-yl-methanone;
[1-(4-Bromo-3-nitro-benzyl)-piperidin-4-yl]-oxazol-2-yl-methanone;
[1-(4-Bromo-2-fluoro-benzyl)-piperidin-4-yl]-oxazol-2-yl-methanone;
[1-(4-Ethoxy-benzyl)-piperidin-4-yl]-oxazol-2-yl-methanone;
[1-(3-Ethoxy-benzyl)-piperidin-4-yl]-oxazol-2-yl-methanone;
[1-(4-Fluoro-benzyl)-piperidin-4-yl]-oxazol-2-yl-methanone;
[1-(3-Fluoro-benzyl)-piperidin-4-yl]-oxazol-2-yl-methanone;
[1-(2-Fluoro-benzyl)-piperidin-4-yl]-oxazol-2-yl-methanone;
[1-(2-Chloro-benzyl)-piperidin-4-yl]-oxazol-2-yl-methanone;
[1-(3-Chloro-benzyl)-piperidin-4-yl]-oxazol-2-yl-methanone;
[1-(4-Chloro-benzyl)-piperidin-4-yl]-oxazol-2-yl-methanone;
Oxazol-2-yl-[1-(4-trifluoromethoxy-benzyl)-piperidin-4-yl]-methanone;
Oxazol-2-yl-[1-(3-trifluoromethoxy-benzyl)-piperidin-4-yl]-methanone;
[1-(3,4-Difluoro-benzyl-piperidin-4-yl)-oxazol-2-yl-methanone;
[1-(1-Methyl-1H-indol-5-ylmethyl)-piperidin-4-yl)-oxazol-2-yl-methanone;
[1-(1-Methyl-1H-indol-6-ylmethyl)-piperidin-4-yl]-oxazol-2-yl-methanone;
(1-[1,8]Naphthyridin-2-ylmethyl-piperidin-4-yl)-oxazol-2-yl-methanone;
[1-(1H-Indol-5-ylmethyl)-piperidin-4-yl]-oxazol-2-yl-methanone;
[1-(3,4-Dimethoxy-benzyl)-piperidin-4-yl]-oxazol-2-yl-methanone;
[1-(3,4-Dichloro-benzyl)-piperidin-4-yl]-oxazol-2-yl-methanone;
[1-(1H-Indol-6-ylmethyl)-piperidin-4-yl]-oxazol-2-yl-methanone;
[1-(6-Methoxy-naphthalen-2-ylmethyl)-piperidin-4-yl]-oxazol-2-yl-methanone;
[1-(1-Hydroxy-naphthalen-2-ylmethyl)-piperidin-4-yl]-oxazol-2-yl-methanone;
(1-Naphthalen-1-ylmethyl-piperidin-4-yl)-oxazol-2-yl-methanone;
[1-(1-Methyl-1H-benzoimidazol-2-ylmethyl)-piperidin-4-yl]-oxazol-2-yl-methanone;
[1-(1-Methyl-1H-indol-2-ylmethyl)-piperidin-4-yl]-oxazol-2-yl-methanone;
[1-(4-Bromo-benzyl)-piperidin-4-yl]-oxazol-2-yl-methanone;
[1-(3-Bromo-benzyl)-piperidin-4-yl]-oxazol-2-yl-methanone;
[1-(2-Bromo-benzyl)-piperidin-4-yl]-oxazol-2-yl-methanone;
(1-Benzo[b]thiophen-2-ylmethyl-piperidin-4-yl)-oxazol-2-yl-methanone;
(1-Benzofuran-2-ylmethyl-piperidin-4-yl)-oxazol-2-yl-methanone;
Oxazol-2-yl-[1-(3-phenoxy-benzyl)-piperidin-4-yl]-methanone;
Oxazol-2-yl-[1-(4-phenoxy-benzyl)-piperidin-4-yl]-methanone;
Oxazol-2-yl-(1-pyridin-4-ylmethyl-piperidin-4-yl)-methanone;
Oxazol-2-yl-(1-pyridin-3-ylmethyl-piperidin-4-yl)-methanone;
Oxazol-2-yl-(1-pyridin-2-ylmethyl-piperidin-4-yl)-methanone;
[1-(4-Methoxy-benzyl)-piperidin-4-yl]-oxazol-2-yl-methanone;
[1-(3-Methoxy-benzyl)-piperidin-4-yl]-oxazol-2-yl-methanone;
[1-(2-Methoxy-benzyl)-piperidin-4-yl]-oxazol-2-yl-methanone;
[1-(4-Methyl-benzyl)-piperidin-4-yl]-oxazol-2-yl-methanone;
[1-(3-Methyl-benzyl)-piperidin-4-yl]-oxazol-2-yl-methanone;
[1-(2-Methyl-benzyl)-piperidin-4-yl]-oxazol-2-yl-methanone;
[1-(6-Chloro-[1,3]dioxolo[4,5-g]quinolin-7-ylmethyl)-piperidin-4-yl]-oxazol-2-yl-methanone;
[1-(2-Chloro-7-methoxy-quinolin-3-ylmethyl)-piperidin-4-yl]-oxazol-2-yl-methanone;
[1-(2-Chloro-7-methyl-quinolin-3-ylmethyl)-piperidin-4-yl]-oxazol-2-yl-methanone;
[1-(2,7-Dichloro-quinolin-3-ylmethyl)-piperidin-4-yl]-oxazol-2-yl-methanone;
[1-(2,2-Difluoro-benzo[1,3]dioxol-5-ylmethyl)-piperidin-4-yl]-oxazol-2-yl-methanone;
[1-(2-Dimethylamino-quinolin-3-ylmethyl)-piperidin-4-yl]-oxazol-2-yl-methanone;
[1-(2-Chloro-8-methyl-quinolin-3-ylmethyl)-piperidin-4-yl]-oxazol-2-yl-methanone;
[1-(2-Chloro-6-methyl-quinolin-3-ylmethyl)-piperidin-4-yl]-oxazol-2-yl-methanone;
[1-(8-Chloro-quinolin-2-ylmethyl)-piperidin-4-yl]-oxazol-2-yl-methanone;
[1-(7-Chloro-quinolin-2-ylmethyl)-piperidin-4-yl]-oxazol-2-yl-methanone;
[1-(6-Chloro-quinolin-2-ylmethyl)-piperidin-4-yl]-oxazol-2-yl-methanone;
Oxazol-2-yl-(1-quinoxalin-2-ylmethyl-piperidin-4-yl)-methanone;
[1-(8-Hydroxy-quinolin-2-ylmethyl)-piperidin-4-yl]-oxazol-2-yl-methanone;
[1-(3-Chloro-quinolin-2-ylmethyl)-piperidin-4-yl]-oxazol-2-yl-methanone;
[1-(6-Methoxy-pyridin-3-ylmethyl)-piperidin-4-yl]-oxazol-2-yl-methanone;
[1-(6-Chloro-pyridin-3-ylmethyl)-piperidin-4-yl]-oxazol-2-yl-methanone;
[1-(6-Methyl-pyridin-2-ylmethyl)-piperidin-4-yl]-oxazol-2-yl-methanone;
Oxazol-2-yl-(1-quinolin-2-ylmethyl-piperidin-4-yl)-methanone;
Oxazol-2-yl-(1-quinolin-3-ylmethyl-piperidin-4-yl)-methanone;
[1-(4-Isopropyl-benzyl)-piperidin-4-yl]-oxazol-2-yl-methanone;
[1-(3,4-Dibromo-benzyl)-piperidin-4-yl]-oxazol-2-yl-methanone;
(1-Naphthalen-2-ylmethyl-piperidin-4-yl)-oxazol-2-yl-methanone;
(1-Benzyl-piperidin-4-yl)-oxazol-2-yl-methanone;
Oxazol-2-yl-[1-(1-phenyl-ethyl)-piperidin-4-yl]-methanone; and
Oxazol-2-yl-{1-[1-(3-trifluoromethyl-phenyl)-ethyl]-piperidin-4-yl}-methanone;
and pharmaceutically acceptable salts thereof.

21. A compound or pharmaceutically acceptable salt according to claim 1.

22. A pharmaceutical composition for treating a disease, disorder, or medical condition mediated by FAAH activity, comprising:
(a) an effective amount of at least one chemical entity selected from compounds of Formula (I): 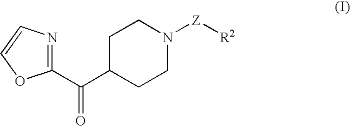
wherein:
Z is —C(O)(CH₂)ₙ—, —SO₂—, or —CH(R^{f})—;
where n is 0, 1, or 2; and
R^{f} is H or C₁₋₄alkyl; and
R² is:
(a) a phenyl group, unsubstituted or substituted with one, two, or three R^{a} moieties;
where each R^{a} moiety is: independently selected from the group consisting of C₁₋₇alkyl, —C₃₋₇cycloalkyl, —C₂₋₇alkenyl, —OH, —OC₃₋₇cycloalkyl, phenyl unsubstituted or substituted with R^{b}, phenoxy unsubstituted or substituted with R^{b}, halo, —CF₃, —OCF₃, —SC₁₋₄alkyl, —SO₂C₁₋₄alkyl, —SOC₁₋₄alkyl, —CN, —NO₂, —CO₂C₁₄alkyl, —CO₂H, —COC₁₋₄alkyl, —SO₂NR^{c}R^{d}, —NR^{c}SO₂R^{d}, —C(O)NR^{c}R^{d}, —NR^{c}C(O)R^{d}, and —N(R^{c})R^{d}; or two adjacent R^{a} moieties together form —O(CH₂)₁₋₂O— or —O(CF₂)O—;
where R^{b} is selected from the group consisting of —C₁₋₄alkyl, —OC₁₋₄alkyl, halo, —CN, —OH, —CF₃, —OCF₃, and —NO₂; and
where R^{c} and R^{d} are each independently —H or —C₁₋₇alkyl;
(b) a five- or six-membered monocyclic heteroaryl group, unsubstituted or substituted with one or two R^{a} moieties;
(c) a naphthyl group, unsubstituted or substituted with one or two R^{e} moieties;
where each R^{e} moiety is: independently selected from the group consisting of —C₁₋₄alkyl, —OC₁₋₄alkyl, halo, —CN, —OH, —CF₃, —OCF₃, and —NO₂; or two adjacent R^{e} moieties together form —O(CH₂)₁₋₂O— or —O(CF₂)O—; or
(d) a nine- or ten-membered fused bicyclic heteroaryl group, unsubstituted or substituted with one or two R^{e} moieties;
and pharmaceutically acceptable salts of Formula (I), pharmaceutically acceptable prodrugs of Formula (I), and pharmaceutically active metabolites of Formula (I); and
(b) a pharmaceutically acceptable excipient.

23. A pharmaceutical composition according to claim 22, wherein said chemical entity is selected from the group consisting of:
[1-(Naphthalene-2-sulfonyl)-piperidin-4-yl]-oxazol-2-yl-methanone;
[1-(4-Methoxy-benzenesulfonyl)-piperidin-4-yl]-oxazol-2-yl-methanone;
[1-(3-Methoxy-benzenesulfonyl)-piperidin-4-yl]-oxazol-2-yl-methanone;
[1-(4-Chloro-benzenesulfonyl)-piperidin-4-yl]-oxazol-2-yl-methanone;
[1-(3-Chloro-benzenesulfonyl)-piperidin-4-yl]-oxazol-2-yl-methanone;
[1-(2-Chloro-benzenesulfonyl)-piperidin-4-yl]-oxazol-2-yl-methanone;
[1-(3-Methoxy-benzoyl)-piperidin-4-yl]-oxazol-2-yl-methanone;
[1-(2-Methoxy-benzoyl)-piperidin-4-yl]-oxazol-2-yl-methanone;
[1-(4-Methoxy-benzoyl)-piperidin-4-yl]-oxazol-2-yl-methanone;
[1-(Naphthalene-2-carbonyl)-piperidin-4-yl]-oxazol-2-yl-methanone;
[1-(4-Chloro-benzoyl)-piperidin-4-yl]-oxazol-2-yl-methanone;
[1-(3-Chloro-benzoyl)-piperidin-4-yl]-oxazol-2-yl-methanone;
[1-(2-Chloro-benzoyl)-piperidin-4-yl]-oxazol-2-yl-methanone;
1-[4-(Oxazole-2-carbonyl)-piperidin-1-yl]-3-phenyl-propan-1-one;
(1-Benzo[1,3]dioxol-5-ylmethyl-piperidin-4-yl)-oxazol-2-yl-methanone;
[1-(3-Isobutoxy-benzyl)-piperidin-4-yl]-oxazol-2-yl-methanone;
[1-(4-Isobutoxy-benzyl)-piperidin-4-yl]-oxazol-2-yl-methanone;
Oxazol-2-yl-[1-(3-trifluoromethyl-benzyl)-piperidin-4-yl]-methanone;
Oxazol-2-yl-[1-(4-trifluoromethyl-benzyl)-piperidin-4-yl]-methanone;
[1-(4-Dimethylamino-benzyl)-piperidin-4-yl]-oxazol-2-yl-methanone;
[1-(4-Cyclohexyloxy-benzyl)-piperidin-4-yl]-oxazol-2-yl-methanone;
[1-(3-Cyclohexyloxy-benzyl)-piperidin-4-yl]-oxazol-2-yl-methanone;
[1-(4-Isopropoxy-benzyl)-piperidin-4-yl]-oxazol-2-yl-methanone;
[1-(3-Isopropoxy-benzyl)-piperidin-4-yl]-oxazol-2-yl-methanone;
[1-(4-Bromo-2-methanesulfonyl-benzyl)-piperidin-4-yl]-oxazol-2-yl-methanone;
[1-(4-Bromo-3-nitro-benzyl)-piperidin-4-yl]-oxazol-2-yl-methanone;
[1-(4-Bromo-2-fluoro-benzyl)-piperidin-4-yl]-oxazol-2-yl-methanone;
[1-(4-Ethoxy-benzyl)-piperidin-4-yl]-oxazol-2-yl-methanone;
[1-(3-Ethoxy-benzyl)-piperidin-4-yl]-oxazol-2-yl-methanone;
[1-(4-Fluoro-benzyl)-piperidin-4-yl]-oxazol-2-yl-methanone;
[1-(3-Fluoro-benzyl)-piperidin-4-yl]-oxazol-2-yl-methanone;
[1-(2-Fluoro-benzyl)-piperidin-4-yl]-oxazol-2-yl-methanone;
[1-(2-Chloro-benzyl)-piperidin-4-yl]-oxazol-2-yl-methanone;
[1-(3-Chloro-benzyl)-piperidin-4-yl]-oxazol-2-yl-methanone;
[1-(4-Chloro-benzyl)-piperidin-4-yl]-oxazol-2-yl-methanone;
Oxazol-2-yl-[1-(4-trifluoromethoxy-benzyl)-piperidin-4-yl]-methanone;
Oxazol-2-yl-[1-(3-trifluoromethoxy-benzyl)-piperidin-4-yl]-methanone;
[1-(3,4-Difluoro-benzyl)-piperidin-4-yl]-oxazol-2-yl-methanone;
[1-(1-Methyl-1H-indol-5-ylmethyl)-piperidin-4-yl]-oxazol-2-yl-methanone;
[1-(1-Methyl-1H-indol-6-ylmethyl)-piperidin-4-yl]-oxazol-2-yl-methanone;
(1-[1,8]Naphthyridin-2-ylmethyl-piperidin-4-yl)-oxazol-2-yl-methanone;
[1-(1H-Indol-5-ylmethyl)-piperidin-4-yl]-oxazol-2-yl-methanone;
[1-(3,4-Dimethoxy-benzyl)-piperidin-4-yl]-oxazol-2-yl-methanone;
[1-(3,4-Dichloro-benzyl)-piperidin-4-yl]-oxazol-2-yl-methanone;
[1-(1H-Indol-6-ylmethyl)-piperidin-4-yl]-oxazol-2-yl-methanone;
[1-(6-Methoxy-naphthalen-2-ylmethyl)-piperidin-4-yl]-oxazol-2-yl-methanone;
[1-(1-Hydroxy-naphthalen-2-ylmethyl)-piperidin-4-yl]-oxazol-2-yl-methanone;
(1-Naphthalen-1-ylmethyl-piperidin-4-yl)-oxazol-2-yl-methanone;
[1-(1-Methyl-1H-benzoimidazol-2-ylmethyl)-piperidin-4-yl]-oxazol-2-yl-methanone;
[1-(1-Methyl-1H-indol-2-ylmethyl)-piperidin-4-yl]-oxazol-2-yl-methanone;
[1-(4-Bromo-benzyl)-piperidin-4-yl]-oxazol-2-yl-methanone;
[1-(3-Bromo-benzyl)-piperidin-4-yl]-oxazol-2-yl-methanone;
[1-(2-Bromo-benzyl)-piperidin-4-yl]-oxazol-2-yl-methanone;
(1-Benzo[b]thiophen-2-ylmethyl-piperidin-4-yl)-oxazol-2-yl-methanone;
(1-Benzofuran-2-ylmethyl-piperidin-4-yl)-oxazol-2-yl-methanone;
Oxazol-2-yl-[1-(3-phenoxy-benzyl)-piperidin-4-yl]-methanone;
Oxazol-2-yl-[1-(4-phenoxy-benzyl)-piperidin-4-yl]-methanone;
Oxazol-2-yl-(1-pyridin-4-ylmethyl-piperidin-4-yl)-methanone;
Oxazol-2-yl-(1-pyridin-3-ylmethyl-piperidin-4-yl)-methanone;
Oxazol-2-yl-(1-pyridin-2-ylmethyl-piperidin-4-yl)-methanone;
[1-(4-Methoxy-benzyl)-piperidin-4-yl]-oxazol-2-yl-methanone;
[1-(3-Methoxy-benzyl)-piperidin-4-yl]-oxazol-2-yl-methanone;
[1-(2-Methoxy-benzyl)-piperidin-4-yl]-oxazol-2-yl-methanone;
[1-(4-Methyl-benzyl)-piperidin-4-yl]-oxazol-2-yl-methanone;
[1-(3-Methyl-benzyl)-piperidin-4-yl]-oxazol-2-yl-methanone;
[1-(2-Methyl-benzyl)-piperidin-4-yl]-oxazol-2-yl-methanone;
[1-(6-Chloro-[1,3]dioxolo[4,5-g]quinolin-7-ylmethyl)-piperidin-4-yl]-oxazol-2-yl-methanone;
[1-(2-Chloro-7-methoxy-quinolin-3-ylmethyl)-piperidin-4-yl]-oxazol-2-yl-methanone;
[1-(2-Chloro-7-methyl-quinolin-3-ylmethyl)-piperidin-4-yl]-oxazol-2-yl-methanone;
[1-(2,7-Dichloro-quinolin-3-ylmethyl)-piperidin-4-yl]-oxazol-2-yl-methanone;
[1-(2,2-Difluoro-benzo[1,3]dioxol-5-ylmethyl)-piperidin-4-yl]-oxazol-2-yl-methanone;
[1-(2-Dimethylamino-quinolin-3-ylmethyl)-piperidin-4-yl]-oxazol-2-yl-methanone;
[1-(2-Chloro-8-methyl-quinolin-3-ylmethyl)-piperidin-4-yl]-oxazol-2-yl-methanone;
[1-(2-Chloro-6-methyl-quinolin-3-ylmethyl)-piperidin-4-yl]-oxazol-2-yl-methanone;
[1-(8-Chloro-quinolin-2-ylmethyl)-piperidin-4-yl]-oxazol-2-yl-methanone;
[1-(7-Chloro-quinolin-2-ylmethyl)-piperidin-4-yl]-oxazol-2-yl-methanone;
[1-(6-Chloro-quinolin-2-ylmethyl)-piperidin-4-yl]-oxazol-2-yl-methanone;
Oxazol-2-yl-(1-quinoxalin-2-ylmethyl-piperidin-4-yl)-methanone;
[1-(8-Hydroxy-quinolin-2-ylmethyl)-piperidin-4-yl]-oxazol-2-yl-methanone;
[1-(3-Chloro-quinolin-2-ylmethyl)-piperidin-4-yl]-oxazol-2-yl-methanone;
[1-(6-Methoxy-pyridin-3-ylmethyl)-piperidin-4-yl]-oxazol-2-yl-methanone;
[1-(6-Chloro-pyridin-3-ylmethyl)-piperidin-4-yl]-oxazol-2-yl-methanone;
[1-(6-Methyl-pyridin-2-ylmethyl)-piperidin-4-yl]-oxazol-2-yl-methanone;
Oxazol-2-yl-(1-quinolin-2-ylmethyl-piperidin-4-yl)-methanone;
Oxazol-2-yl-(1-quinolin-3-ylmethyl-piperidin-4-yl)-methanone;
[1-(4-Isopropyl-benzyl)-piperidin-4-yl]-oxazol-2-yl-methanone;
[1-(3,4-Dibromo-benzyl)-piperidin-4-yl]-oxazol-2-yl-methanone;
(1-Naphthalen-2-ylmethyl-piperidin-4-yl)-oxazol-2-yl-methanone;
(1-Benzyl-piperidin-4-yl)-oxazol-2-yl-methanone;
Oxazol-2-yl-[1-(1-phenyl-ethyl)-piperidin-4-yl]-methanone; and
Oxazol-2-yl-{1-[1-(3-trifluoromethyl-phenyl)-ethyl]-piperidin-4-yl}-methanone;
and pharmaceutically acceptable salts thereof.

24. A pharmaceutical composition according to claim 22, further comprising: an analgesic selected from the group consisting of opioids and non-steroidal anti-inflammatory drugs.

25. A pharmaceutical composition according to claim 22, further comprising: an additional active ingredient selected from the group consisting of aspirin, acetaminophen, opioids, ibuprofen, naproxen, COX-2 inhibitors, gabapentin, pregabalin, and tramadol.

26. A method of treating a subject suffering from or diagnosed with a disease, disorder, or medical condition mediated by FAAH activity, comprising administering to the subject in need of such treatment an effective amount of at least one chemical entity selected from compounds of Formula (I): 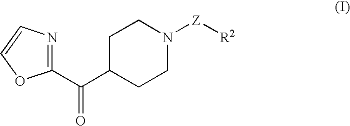
wherein:
Z is —C(O)(CH₂)ₙ—, —SO₂—, or —CH(R^{f})—;
where n is 0, 1, or 2; and
R^{f} is H or C₁-alkyl; and
R² is:
(a) a phenyl group, unsubstituted or substituted with one, two, or three R^{a} moieties;
where each R^{a} moiety is: independently selected from the group consisting of C₁₋₇alkyl, —C₃₋₇cycloalkyl, —C₂₋₇alkenyl, —OH, —OC₃₋₇cycloalkyl, phenyl unsubstituted or substituted with R^{b}, phenoxy unsubstituted or substituted with R^{b}, halo, —CF₃, —OCF₃, —SC₁₋₄alkyl, —SO₂C₁₋₄alkyl, —SOC₁₋₄alkyl, —CN, —NO₂, —CO₂H, —SO₂NR^{c}R^{d}, —NR^{c}SO₂R^{d}, —C(O)NR^{c}R^{d}, —NR^{c}C(O)R^{d}, and —N(R^{c})R^{d}; or two adjacent R^{a} moieties together form —O(CH₂)₁₋₂O— or —O(CF₂)O—;
where R^{b} is selected from the group consisting of —C₁₋₄alkyl, halo, —CN, —OH, —CF₃, —OCF₃, and —NO₂; and
where R^{c} and R^{d} are each independently —H or —C₁₋₇alkyl;
(b) a five- or six-membered monocyclic heteroaryl group, unsubstituted or substituted with one or two R^{a} moieties;
(c) a naphthyl group, unsubstituted or substituted with one or two R^{e} moieties; where each R^{e} moiety is: independently selected from the group consisting of —C₁₋₄alkyl, —OC₁₋₄alkyl, halo, —CN, —OH, —CF₃, —OCF₃, and —NO₂; or two adjacent R^{e} moieties together form —O(CH₂)₁₋₂O— or —O(CF₂)O—; or
(d) a nine- or ten-membered fused bicyclic heteroaryl group, unsubstituted or substituted with one or two R^{e} moieties;
and pharmaceutically acceptable salts of Formula (I), pharmaceutically acceptable prodrugs of Formula (I), and pharmaceutically active metabolites of Formula (I).

27. A method according to claim 26, wherein said chemical entity is selected from the group consisting of:
[1-(Naphthalene-2-sulfonyl)-piperidin-4-yl]-oxazol-2-yl-methanone;
[1-(4-Methoxy-benzenesulfonyl)-piperidin-4-yl]-oxazol-2-yl-methanone;
[1-(3-Methoxy-benzenesulfonyl)-piperidin-4-yl]-oxazol-2-yl-methanone;
[1-(4-Chloro-benzenesulfonyl)-piperidin-4-yl]-oxazol-2-yl-methanone;
[1-(3-Chloro-benzenesulfonyl)-piperidin-4-yl]-oxazol-2-yl-methanone;
[1-(2-Chloro-benzenesulfonyl)-piperidin-4-yl]-oxazol-2-yl-methanone;
[1-(3-Methoxy-benzoyl)-piperidin-4-yl]-oxazol-2-yl-methanone;
[1-(2-Methoxy-benzoyl)-piperidin-4-yl]-oxazol-2-yl-methanone;
[1-(4-Methoxy-benzoyl)-piperidin-4-yl]-oxazol-2-yl-methanone;
[1-(Naphthalene-2-carbonyl)-piperidin-4-yl]-oxazol-2-yl-methanone;
[1-(4-Chloro-benzoyl)-piperidin-4-yl]-oxazol-2-yl-methanone;
[1-(3-Chloro-benzoyl)-piperidin-4-yl]-oxazol-2-yl-methanone;
[1-(2-Chloro-benzoyl)-piperidin-4-yl]-oxazol-2-yl-methanone;
1-[4-(Oxazole-2-carbonyl)-piperidin-1-yl]-3-phenyl-propan-1-one;
(1-Benzo[1,3]dioxol-5-ylmethyl-piperidin-4-yl)-oxazol-2-yl-methanone;
[1-(3-Isobutoxy-benzyl)-piperidin-4-yl]-oxazol-2-yl-methanone;
[1-(4-Isobutoxy-benzyl)-piperidin-4-yl]-oxazol-2-yl-methanone;
Oxazol-2-yl-[1-(3-trifluoromethyl-benzyl)-piperidin-4-yl]-methanone;
Oxazol-2-yl-[1-(4-trifluoromethyl-benzyl)-piperidin-4-yl]-methanone;
[1-(4-Dimethylamino-benzyl)-piperidin-4-yl]-oxazol-2-yl-methanone;
[1-(4-Cyclohexyloxy-benzyl)-piperidin-4-yl]-oxazol-2-yl-methanone;
[1-(3-Cyclohexyloxy-benzyl)-piperidin-4-yl]-oxazol-2-yl-methanone;
[1-(4-Isopropoxy-benzyl)-piperidin-4-yl]-oxazol-2-yl-methanone;
[1-(3-Isopropoxy-benzyl)-piperidin-4-yl]-oxazol-2-yl-methanone;
[1-(4-Bromo-2-methanesulfonyl-benzyl)-piperidin-4-yl]-oxazol-2-yl-methanone;
[1-(4-Bromo-3-nitro-benzyl)-piperidin-4-yl]-oxazol-2-yl-methanone;
[1-(4-Bromo-2-fluoro-benzyl)-piperidin-4-yl]-oxazol-2-yl-methanone;
[1-(4-Ethoxy-benzyl)-piperidin-4-yl]-oxazol-2-yl-methanone;
[1-(3-Ethoxy-benzyl)-piperidin-4-yl]-oxazol-2-yl-methanone;
[1-(4-Fluoro-benzyl)-piperidin-4-yl]-oxazol-2-yl-methanone;
[1-(3-Fluoro-benzyl)-piperidin-4-yl]-oxazol-2-yl-methanone;
[1-(2-Fluoro-benzyl)-piperidin-4-yl]-oxazol-2-yl-methanone;
[1-(2-Chloro-benzyl)-piperidin-4-yl]-oxazol-2-yl-methanone;
[1-(3-Chloro-benzyl)-piperidin-4-yl]-oxazol-2-yl-methanone;
[1-(4-Chloro-benzyl)-piperidin-4-yl]-oxazol-2-yl-methanone;
Oxazol-2-yl-[1-(4-trifluoromethoxy-benzyl)-piperidin-4-yl]-methanone;
Oxazol-2-yl-[1-(3-trifluoromethoxy-benzyl)-piperidin-4-yl]-methanone;
[1-(3,4-Difluoro-benzyl)-piperidin-4-yl]-oxazol-2-yl-methanone;
[1-(1-Methyl-1H-indol-5-ylmethyl)-piperidin-4-yl]-oxazol-2-yl-methanone;
[1-(1-Methyl-1H-indol-6-ylmethyl)-piperidin-4-yl]-oxazol-2-yl-methanone;
(1-[1,8]Naphthyridin-2-ylmethyl-piperidin-4-yl)-oxazol-2-yl-methanone;
[1-(1H-Indol-5-ylmethyl)-piperidin-4-yl]-oxazol-2-yl-methanone;
[1-(3,4-Dimethoxy-benzyl)-piperidin-4-yl]-oxazol-2-yl-methanone;
[1-(3,4-Dichloro-benzyl)-piperidin-4-yl]-oxazol-2-yl-methanone;
[1-(1H-Indol-6-ylmethyl)-piperidin-4-yl]-oxazol-2-yl-methanone;
[1-(6-Methoxy-naphthalen-2-ylmethyl)-piperidin-4-yl]-oxazol-2-yl-methanone;
[1-(1-Hydroxy-naphthalen-2-ylmethyl)-piperidin-4-yl]-oxazol-2-yl-methanone;
(1-Naphthalen-1-ylmethyl-piperidin-4-yl)-oxazol-2-yl-methanone;
[1-(1-Methyl-1H-benzoimidazol-2-ylmethyl)-piperidin-4-yl]-oxazol-2-yl-methanone;
[1-(1-Methyl-1H-indol-2-ylmethyl)-piperidin-4-yl]-oxazol-2-yl-methanone;
[1-(4-Bromo-benzyl)-piperidin-4-yl]-oxazol-2-yl-methanone;
[1-(3-Bromo-benzyl)-piperidin-4-yl]-oxazol-2-yl-methanone;
[1-(2-Bromo-benzyl)-piperidin-4-yl]-oxazol-2-yl-methanone;
(1-Benzo[b]thiophen-2-ylmethyl-piperidin-4-yl)-oxazol-2-yl-methanone;
(1-Benzofuran-2-ylmethyl-piperidin-4-yl)-oxazol-2-yl-methanone;
Oxazol-2-yl-[1-(3-phenoxy-benzyl)-piperidin-4-yl]-methanone;
Oxazol-2-yl-[1-(4-phenoxy-benzyl)-piperidin-4-yl]-methanone;
Oxazol-2-yl-(1-pyridin-4-ylmethyl-piperidin-4-yl)-methanone;
Oxazol-2-yl-(1-pyridin-3-ylmethyl-piperidin-4-yl)-methanone;
Oxazol-2-yl-(1-pyridin-2-ylmethyl-piperidin-4-yl)-methanone;
[1-(4-Methoxy-benzyl)-piperidin-4-yl]-oxazol-2-yl-methanone;
[1-(3-Methoxy-benzyl)-piperidin-4-yl]-oxazol-2-yl-methanone;
[1-(2-Methoxy-benzyl)-piperidin-4-yl]-oxazol-2-yl-methanone;
[1-(4-Methyl-benzyl)-piperidin-4-yl]-oxazol-2-yl-methanone;
[1-(3-Methyl-benzyl)-piperidin-4-yl]-oxazol-2-yl-methanone;
[1-(2-Methyl-benzyl)-piperidin-4-yl]-oxazol-2-yl-methanone;
[1-(6-Chloro-[1,3]dioxolo[4,5-g]quinolin-7-ylmethyl)-piperidin-4-yl]-oxazol-2-yl-methanone;
[1-(2-Chloro-7-methoxy-quinolin-3-ylmethyl)-piperidin-4-yl]-oxazol-2-yl-methanone;
[1-(2-Chloro-7-methyl-quinolin-3-ylmethyl)-piperidin-4-yl]-oxazol-2-yl-methanone;
[1-(2,7-Dichloro-quinolin-3-ylmethyl)-piperidin-4-yl]-oxazol-2-yl-methanone;
[1-(2,2-Difluoro-benzo[1,3]dioxol-5-ylmethyl)-piperidin-4-yl]-oxazol-2-yl-methanone;
[1-(2-Dimethylamino-quinolin-3-ylmethyl)-piperidin-4-yl]-oxazol-2-yl-methanone;
[1-(2-Chloro-8-meth-quinolin-3-ylmethyl)-piperidin-4-yl]-oxazol-2-yl-methanone;
[1-(2-Chloro-6-methyl-quinolin-3-ylmethyl)-piperidin-4-yl]-oxazol-2-yl-methanone;
[1-(8-Chloro-quinolin-2-ylmethyl)-piperidin-4-yl]-oxazol-2-yl-methanone;
[1-(7-Chloro-quinolin-2-ylmethyl)-piperidin-4-yl]-oxazol-2-yl-methanone;
[1-(6-Chloro-quinolin-2-ylmethyl)-piperidin-4-yl]-oxazol-2-yl-methanone;
Oxazol-2-yl-(1-quinoxalin-2-ylmethyl-piperidin-4-yl)-methanone;
[1-(8-Hydroxy-quinolin-2-ylmethyl)-piperidin-4-yl]-oxazol-2-yl-methanone;
[1-(3-Chloro-quinolin-2-ylmethyl)-piperidin-4-yl]-oxazol-2-yl-methanone;
[1-(6-Methoxy-pyridin-3-ylmethyl)-piperidin-4-yl]-oxazol-2-yl-methanone;
[1-(6-Chloro-pyridin-3-ylmethyl)-piperidin-4-yl]-oxazol-2-yl-methanone;
[1-(6-Methyl-pyridin-2-ylmethyl)-piperidin-4-yl]-oxazol-2-yl-methanone;
Oxazol-2-yl-(1-quinolin-2-ylmethyl-piperidin-4-yl)-methanone;
Oxazol-2-yl-(1-quinolin-3-ylmethyl-piperidin-4-yl)-methanone;
[1-(4-Isopropyl-benzyl)-piperidin-4-yl]-oxazol-2-yl-methanone;
[1-(3,4-Dibromo-benzyl)-piperidin-4-yl]-oxazol-2-yl-methanone;
(1-Naphthalen-2-ylmethyl-piperidin-4-yl)-oxazol-2-yl-methanone;
(1-Benzyl-piperidin-4-yl)-oxazol-2-yl-methanone;
Oxazol-2-yl-[1-(1-phenyl-ethyl)-piperidin-4-yl]-methanone; and
Oxazol-2-yl-{1-[1-(3-trifluoromethyl-phenyl)-ethyl]-piperidin-4-yl}-methanone;
and pharmaceutically acceptable salts thereof.

28. A method according to claim 26, wherein the disease, disorder, or medical condition is selected from the group consisting of: anxiety, depression, pain, sleep disorders, eating disorders, inflammation, movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, and neuroinflammation.

29. A method according to claim 26, wherein the disease, disorder, or medical condition is selected from the group consisting of: anxiety, pain, inflammation, sleep disorders, eating disorders, and movement disorders.

30. A method according to claim 26, wherein the disease, disorder, or medical condition is multiple sclerosis.

31. A method according to claim 26, wherein the disease, disorder, or medical condition is pain or inflammation.
